(19) United States
(12) Patent Application Publication
(10) Pub. No.: US 2026/0345801 A1
(43) Pub. Date: Nov. 5, 2026

(54) METHODS FOR SENSITIZING RESPONSE TO AN IMMUNOTHERAPY

(71) Applicant: The Regents of the University of California, Oakland, CA (US)

(72) Inventors: Fotios Asimakopoulos, San Diego, CA (US); Athanasios Papadas, San Diego, CA (US); Alexander Patrick Cicala, San Diego, CA (US)

(21) Appl. No.: 19/124,135

(22) PCT Filed: Aug. 15, 2023
(86) PCT No.: PCT/US2023/072232

§ 371 Date: Apr. 24, 2025

Related U.S. Application Data
(60) Provisional application No. 63/419,194, filed on Oct. 25, 2022.
(60) Provisional application No. 63/480,173, filed on Jan. 17, 2023.

(51) Int. Cl. A61K 47/64; A61K 31/352; A61K 38/00; A61P 35/00; C07K 14/47; C07K 14/52; C07K 14/535; C07K 14/54; C07K 14/55; C07K 14/57; C07K 16/28; C12N 15/86; G01N 33/68

(52) U.S. Cl. CPC A61K 47/642; A61K 31/352; A61P 35/00; C07K 14/4702; C07K 14/522; C07K 14/523; C07K 14/535; C07K 14/5434; C07K 14/5443; C07K 14/55; C07K 14/57; C07K 16/2818; C07K 16/2827; C07K 16/2878; C12N 15/86; G01N 33/6893; A61K 38/00; C07K 2319/02; C12N 2740/15043; G01N 2333/4722; G01N 2800/52

(57) ABSTRACT

Provided herein are versikine fusions, including versikine fusions with GM-CSF, CCL5, XCL1, FLT3L, CCL7, CXCL9, CXCL10, IL-12, IL-27, IL-15, IL18, IL-23, IL-2, IFN, as well as secretory peptides. Additionally, provided herein are methods for introducing versikine and versikine fusion proteins to cells, in vitro and in vivo. Additionally, provided herein are methods of treating cancer, increasing T cell activation in a tumor microenvironment (TME), or sensitizing a response to an immunotherapy in a subject in need thereof, the method comprising administering a proteolytic fragment of an extracellular matrix (ECM) proteoglycan.

### CROSS-REFERENCE TO RELATED APPLICATIONS

This application claims priority to U.S. Provisional Patent Application No. 63/419,194, filed on Oct. 25, 2022, and U.S. Provisional Patent Application No. 63/480,173, filed on Jan. 17, 2023. The disclosure of the prior applications is considered part of the disclosure of this application and is incorporated herein by reference in its entirety.

### FEDERALLY SPONSORED RESEARCH OR DEVELOPMENT

This invention was made with Government support under Grant No. CA252937 awarded by the National Institutes of Health. The Government has certain rights in the invention.

### SEQUENCE LISTING

This application contains a Sequence Listing that has been submitted electronically as an XML file named “15670-0367WO1.XML.” The XML file, created on Aug. 14, 2023, is 34,725 bytes in size. The material in the XML file is hereby incorporated by reference in its entirety.

### BACKGROUND

T-cell-inflamed tumor microenvironments (TME) are prerequisite for immunotherapy efficacy; however, the drivers for promoting T cell priming and infiltration within the TME remain incompletely understood. Furthermore, conventional type I dendritic cells (cDC1) are indispensable for anti-tumor immunity, wherein several studies have documented the paradoxical localization of cDC1 within tumor stroma.

Tumor antigen cross-presentation and CD8+ T cell effector priming by stimulatory type 1 conventional dendritic cells (cDC1s) is integral to spontaneous and therapeutic anti-tumor immunity. In addition to effector priming in the lymph node and in the tumor microenvironment (TME), cDC1s regulate effector cell influx into the TME From a translational perspective, cDC1s are crucial for responses to vaccination strategies, immune checkpoint inhibitors, and engineered immune effector cells (e.g., chimeric antigen receptor T [CAR-T]cells). It has been shown that stimulatory cDC1s are excluded from interdigitating tumor nestlets and locate in peritumoral stroma. However, the mechanisms that retain cDC1s at the tumor periphery remain poorly understood.

### SUMMARY

Provided herein are fusion proteins comprising a versikine protein having at least 95% identity to SEQ ID NO: 1 and a secretory peptide having a sequence of SEQ ID NO: 2-7.

Also provided herein are fusion proteins comprising a versikine protein having at least 95% identity to SEQ ID NO: 1 and a protein selected from the group consisting of GM-CSF, CCL5, XCL1, FLT3L, CCL7, CXCL9, CXCL10, IL-12, IL-27, IL-15, IL18, IL-23, IL-2, and IFNgamma. In some embodiments, a protein selected from the group consisting of GM-CSF, CCL5, XCL1, FLT3L, CCL7, CXCL9, CXCL10, IL-12, IL-27, IL-15, IL18, IL-23, IL-2, and IFNgamma comprises a sequence selected from the group consisting of SEQ ID NO 8-21. In some embodiments, the fusion protein further comprises a secretory peptide. In some embodiments, the secretory peptide comprises a sequence of SQ ID NO: 2-7.

Also provided herein are vectors comprising any one of the fusion peptides described herein. In some embodiments, the vector is a viral vector. In some embodiments, the viral vector is a lentiviral vector.

Also provided herein are methods of introducing versikine to antigen-presenting cells, the method comprising: introducing to an antigen-presenting cell any one of the fusion proteins or the vectors described herein, wherein the introducing comprises a lentiviral vector. In some embodiments, the antigen-presenting cells comprise macrophages, B cells, fibroblasts, and/or dendritic cells. In some embodiments, the antigen-presenting cell is a cell culture. In some embodiments, the antigen-presenting cell is in a subject.

Also provided herein are conjugated polypeptides comprising a versikine protein having at least 95% sequence identity to SEQ ID NO: 1 and a small molecule STING agonist. In some embodiments, the small molecule STING agonist comprises DMXAA or RVU-27065.

Also provided herein are pharmaceutical compositions comprising a versikine protein having at least 95% identity to SEQ ID NO: 1 and a STING agonist. In some embodiments, the STING agonist is DMXAA or RVU-27065. In some embodiments, the versikine protein is co-administered with the STING agonist. In some embodiments, the versikine protein is administered before the STING agonist.

Also provided herein are pharmaceutical compositions comprising a versikine protein having at least 95% identity to SEQ ID NO: 1 and an anti-PD1 and/or anti-PD-L1 inhibitor. In some embodiments, the anti-PD1 and/or anti-PD-L1 inhibitor comprises pembrolizumab, nivolumab, cemiplimab, JTX-4014, spartalizumab, camrelizumab, sintilimab, tislelizumab, toripalimab, dostarlimab, atezolizumab, avelumab, durvalumab, KN035, CK-301, AUNP12, CA-170, or BMS-986189. In some embodiments, the versikine protein is co-administered with the anti-PD1 and/or anti-PD-L1 inhibitor. In some embodiments, the versikine protein is administered before the anti-PD1 and/or anti-PD-L1 inhibitor.

Also provided herein are pharmaceutical compositions comprising a versikine protein having at least 95% sequence identity to SEQ ID NO: 1 and anti-CD40 immunotherapy. In some embodiments, the anti-CD40 immunotherapy is selected from the group consisting of selicrelumab, APX005M, JNJ-64457107, SEA-CD40, ChiLob7/4, CDX-1140H, Dacetuzumab, and ABBV-428. In some embodiments, the versikine protein is co-administered with the anti-CD40 immunotherapy. In some embodiments, the versikine protein is administered before the anti-CD40 immunotherapy.

Also provided herein are methods of treating cancer, the method comprising administering a versikine protein having at least 95% sequence identity to SEQ ID NO: 1 or a versikine nucleic acid having at least 95% sequence identity to SEQ ID NO: 23.

Also provided herein are methods for increasing T cell activation in a tumor microenvironment (TME) in a subject, the method comprising: administering a versikine protein having at least 95% sequence identity to SEQ ID NO: 1 or a versikine nucleic acid having at least 95% sequence identity to SEQ ID NO: 23.

Also provided herein are methods for sensitizing a response to an immunotherapy in a subject in need thereof, the method comprising: administering a versikine protein having at least 95% sequence identity to SEQ ID NO: 1 or a versikine nucleic acid having at least 95% sequence identity to SEQ ID NO: 23.

In some embodiments, the versikine protein or versikine nucleic acid is co-administered with another pharmaceutical compound. In some embodiments, the pharmaceutical compound comprises a stimulator of interferon genes (STING) agonist immunotherapy. In some embodiments, the pharmaceutical compound comprises an anti-PD1 immunotherapy. In some embodiments, the pharmaceutical compound comprises an anti-CD40 immunotherapy. In some embodiments, the administering comprises delivery of the versikine.

In some embodiments, the cell is a dendritic cell. In some embodiments, the cell is an antigen-presenting cell. In some embodiments, the delivery of the proteolytic fragment comprises a virus-based delivery. In some embodiments, the virus-based delivery comprises a lentivirus.

In some embodiments, the versikine is generated by proteolysis of VCAN at the Glu441-Ala442 bond. In some embodiments, the versikine protein or versikine nucleic acid is conjugated to another protein. In some embodiments, the other protein is selected from the group consisting of: GM-CSF, CCL5, XCL1, FLT3L, CCL7, CXCL9, CXCL10, IL-12, IL-27, IL-15, IL18, IL-23, IL-2, or IFN. In some embodiments, the other protein comprises a secretory peptide. In some embodiments, the secretory peptide is selected from the group consisting of human OSM, human immunoglobulin, human chymotrypsinogen, human trypsinogen 2, human IL-2, or human insulin.

In some embodiments, the versikine protein or versikine nucleic acid is conjugated to a drug. In some embodiments, the drug comprises a small molecule STING agonist. In some embodiments, the versikine protein or versikine nucleic acid is conjugated to a small molecule. In some embodiments, the versikine protein or versikine nucleic acid is conjugated to a radiochemical. In some embodiments, the versikine protein or versikine nucleic acid is conjugated to an immunogenic component. In some embodiments, the versikine protein or versikine nucleic acid is conjugated to a label. In some embodiments, the label is a radioisotope or a fluorophore.

In some embodiments, the versikine is a nucleic acid. In some embodiments, the versikine nucleic acid is fused to non-versikine nucleic acid. In some embodiments, the non-versikine nucleic acid is a DNA or RNA molecule.

In some embodiments, a method provided herein further comprises administering a pharmaceutical agent. In some embodiments, the pharmaceutical agent comprises an immunotherapy agent. In some embodiments, the immunotherapy agent comprises a STING agonist, a PD-1 inhibitor, a PD-L1 inhibitor, or a CD40 agonist.

In some embodiments, the subject has a tumor. In some embodiments, the tumor is a refractory tumor.

Also provided herein are methods of predicting a subject's response to an immunotherapy, the method comprising: (a) analyzing a biological sample obtained from the subject; and (b) detecting a proteolytic fragment of an extracellular matrix (ECM) proteoglycan in the biological sample, thereby predicting the subject's response to the immunotherapy. In some embodiments, the analyzing comprises staining the biological sample. In some embodiments, the staining comprises an immunofluorescence stain. In some embodiments, the staining comprises an immunohistochemistry stain. In some embodiments, the biological sample is stained with an antibody. In some embodiments, the antibody is selected from the group consisting of: an anti-DPEAAE neo-epitope antibody, anti-HA antibody, anti-XCR1 antibody, anti-CLEC9A antibody, anti-NCR1 antibody, and anti-CD8 antibody. In some embodiments, the detecting comprises determining the level of the proteolytic fragment by measuring the antibody in the biological sample.

In some embodiments, the immunotherapy comprises a stimulator of interferon genes (STING) agonist immunotherapy. In some embodiments, the immunotherapy comprises an anti-PD1 immunotherapy. In some embodiments, the immunotherapy comprises an anti-CD40 immunotherapy.

In some embodiments, the ECM proteoglycan is versican (VCAN). In some embodiments, the proteolytic fragment of the ECM proteoglycan is versikine. In some embodiments, the versikine is generated by proteolysis of VCAN at the Glu441-Ala442 bond.

In some embodiments, the subject has a tumor. In some embodiments, the tumor is a refractory tumor.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1J show that the VCAN pathway regulates tumor cDC1s. FIG. 1A shows an exemplary schematic showing versican (VCAN)-V1 functional domains and site-specific proteolysis to generate versikine (scissors represent ADAMTS proteolytic cleavage). CS, chondroitin sulphate. FIG. 1B shows stromal distribution of anti-DPEAAE IHC staining in human lung cancers. DPEAAE constitutes the C terminus of versikine (chromogen, DAB; counterstain, hematoxylin). FIG. 1C shows triple IHC staining of human lung cancers (DPEAAE, teal; XCR1, brown; CD8, purple). FIG. 1D shows distribution of VCAN expression across TCGA carcinomas, ordered on the horizontal axis by median VCAN expression. FIG. 1E shows distribution of cDC1 (BATF3-DC) score across TCGA carcinomas, ordered on the horizontal axis by median measured cDC1 score. FIG. 1F shows levels of correlation between cDC1 (BATF3-DC) score and VCAN expression across TCGA carcinomas. The ranked median of VCAN expression and measured cDC1 (BATF3-DC) score is shown across the x axis (1, highest; 20, lowest). Significant (q<0.1) correlations after multiple hypothesis correction are colored red. Error bars represent the standard error of the correlation coefficient measured using Python statsmodels. FIG. 1G shows generation of Vcan^{+/-} mice through CRISPR-Cas9-based targeting of Vcan exon 3. FIG. 1H shows mass cytometry of CD45+ cells from WT (LLC implanted into WT recipients, left) and Vcan-depleted tumors (LLC^{VcanKD} tumor cells implanted into Vcan^{-/-} recipients, right). FIG. 1I shows quantification of frequency (left) and absolute count ratios (cDC1/cDC1+cDC2 and cDC2/cDC1+cDC2) in WT, Vcan-depleted (LLC-EV^{VcanKD}:Vcan^{+/-}), and versikine (Vkine)-rescued (LLC-Vkine^{VcanKD}:Vcan^{+/-}) tumors. Data are presented as mean±SEM. n=5 for each group. *p<0.05, **p<0.01, ***p<0.001. FIG. 1J shows representative flow cytometry plots showing cDC1 and cDC2 frequency in WT, Vcan-depleted (LLC-EV^{VcanKD}:Vcan^{+/-}), and Vkine-rescued (LLC-Vkine^{VcanKD}:Vcan^{+/-}) tumors. FIG. 1K shows human tonsil immunohistochemical staining for cDC1 lineage markers XCR1 and CLEC9A. Left, composite image after spectral unmixing. XCR1 (DAB), CLEC9A. Right, scoring map: double-negative; single-positive XCR1; single-positive CLEC9A; double-positive XCR1/CLEC9A. 23 different areas were imaged, each containing approximately 4173 cells. About 80% of cells had no expression of markers and ~14% had dual staining. FIG. 1L shows an exemplary schematic depiction of the deletions in the two mutant Vcan founders, 1053 (16 bp deletion) and 1058 (47 bp deletion). Sequence of exon 3 primer used in RT-PCR experiments is shown. FIG. 1M shows DNA amplification using primers flanking the targeted region. Shown are a 128 bp WT amplicon and the mutated amplicons in founders Vcan1053 and Vcan1058. FIG. 1N shows bone marrow-derived macrophage (BMDM) Vcan locus RT-PCR using exon 3 primers at baseline or after stimulation with TLR4 agonist, lipopolysaccharide (LPS). Vcan1053 demonstrates a more severe defect in Vcan message induction/stability than Vcan1058. FIG. 1O shows validation of Vcan knockdown in LLC^{VcanKD} cells. LLC cells were transfected with each of 3 hairpins (shRNA #1, 2 or 3) targeting exon 8 (encoding GAGD domain depicted in FIG. 1A). Vcan message was assayed using exon 3 primers (left) and exon 15 primers (right). FIG. 1P shows gating strategy to delineate tumor-associated dendritic cells (TADC) per van Ginderachter schema. FIG. 1Q shows cDC subset frequencies in steady-state splenic tissue from WT and Vcan−/− mice. FIG. 1R shows total cDC (cDC1+cDC2) absolute counts per mg tumor mass in LLC:WT, Vcan-depleted (LLC-EV^{VcanKD}:Vcan+/−) and versikine-rescue (LLC-Vkine^{VcanKD}:Vcan+/−) tumors, using the Collagenase/Hyaluronidase tumor cell dissociation protocol. FIG. 1S shows growth rates of WT, Vcan-depleted (LLC-EV^{VcanKD}:Vcan+/−) and versikine-rescue (LLCVkine^{VcanKD}:Vcan+/−) tumors. In FIGS. 1R and 1S, the colon separates the genotype of implanted tumor cells from genotype of recipient animal.

FIGS. 2A-2G show that the VCAN-matrikine versikine promotes cDC1 abundance in vivo. FIG. 2A shows an exemplary schematic of the experiment. LLC tumor cells that were engineered to express hemagglutinin (HA)-tagged versikine (LLC-Vkine) or empty vector controls (LLC-EV) and injected subcutaneously (s.c.) on the flank or intravenously using a retro-orbital approach.

FIG. 2B shows gross morphology of orthotopic (top) and s.c. (bottom) LLC-EV and LLC-Vkine tumors. FIG. 2C shows anti-HA tag western blotting detects a 75-kDa band in LLC-Vkine tumor lysates, consistent with versikine. FIG. 2D shows representative immunohistochemistry (IHC) images showing α-DPEAAE and HA tag staining of LLC-EV and LLC-Vkine tumors. Endogenous DPEAAE proteolysis is low level and similar between LLC-EV and LLC-Vkine. Anti-HA staining localizes in a membranous distribution in LLC-Vkine cells (inset, larger magnification). FIG. 2E shows flow cytometric analysis of cDC subsets in s.c. LLC-EV and LLC-Vkine tumors and quantification of cDC and tumor-associated DC (TADC) frequency (top) and absolute count ratios (cDC1/cDC1+cDC2 and cDC2/cDC1+cDC2) (bottom). FIG. 2F shows comparison of immune contexture (CD45+ fraction) in LLC-EV versus LLC-Vkine tumors by 31-marker mass cytometry. FIG. 2G shows flow cytometry analysis of cDC subsets in orthotopic LLC-EV and LLC-Vkine tumors (lung metastases induced by intravenous injection). A summary of cDC and TADC subset frequencies is depicted on the right. FIG. 2H shows full-length western blot for FIG. 2C. Arrow, versikine band migrating at 75 KDa. FIG. 2I shows example of human lung cancer biopsy with stromal plus epithelial DPEAAE staining. 10× objective: scalebar 240 mm, 40× objective: scalebar 60 mm. FIG. 2J shows examples of negative DPEAAE staining in human lung cancer biopsies. 10× objective: scalebar 240 mm, 40× objective: scalebar 60 mm. FIG. 2K shows membranous localization of HAtagged ectopic versikine in B16 melanoma, a tumor model characterized by absence of cellautonomous Vcan expression. HA-tag, chromogen: BCIP/NBT; counterstain:nuclear fast red. FIG. 2L shows growth rates of subcutaneous LLC-EV and LLC-Vkine tumors. FIG. 2M shows absolute counts (cell count/mg of tumor) of major intratumoral DC subsets, following optimized cell dissociation protocols (Miltenyi Dissociation Kit). FIG. 2N shows flow cytometric analysis of cDC subsets in orthotopically-implanted 4T1 mammary carcinoma tumors engineered to express empty-vector (4T1-EV) or versikine (4T1-Vkine). Representative flow plots (left) and frequencies (right) of cDC subsets are shown. FIG. 2O shows growth rates of orthotopic 4T1-EV and 4T1-Vkine tumors. FIG. 2P shows flow cytometric analysis of cDC subsets from bone marrow following intracardiac injection of VQ myeloma cells, engineered to express empty-vector (VQ-EV) or versikine (VQ-Vkine). Representative flow plots (left) and frequencies (right) of cDC subsets are shown. FIG. 2Q shows Kaplan-Meier curves depicting time-to-hindlimb paralysis (a clinical sequela of myeloma progression) in recipients of VQ-EV vs. VQ-Vkine myeloma tumors.

FIGS. 3A-3G show that versikine selectively activates cDC1 in vivo. FIG. 3A shows RT-PCR analysis for cDC1 “signature” transcripts in bulk LLC-EV and LLC-Vkine tumor mRNA. Data are presented as mean±SEM. FIG. 3B shows summary of CD40 staining intensity (MFI, mean fluorescence intensity) in DC subsets from LLC-EV and LLC-Vkine tumors (experiment 1). Examples of individual histogram plots for each DC subset are shown. FIG. 3C shows a summary of PD-L1 staining intensity in DC subsets from LLC-EV and LLC-Vkine tumors. Examples of individual histogram plots for each DC subset are shown. FIG. 3D shows a layout of the experiment to compare transcriptomic profiles in LLC-EV versus LLC-Vkine tumor immune infiltrates. FIG. 3E shows hierarchical clustering of transcriptomic profiles by RNA sequencing (RNA-seq) analysis of CD45+ tumor-infiltrating leukocytes (TILs) extracted from LLC-EV versus LLC-Vkine tumors. FIG. 3F shows volcano plot highlighting key differentially expressed genes in CD45+ TILs from LLC-Vkine tumors compared with LLC-EV tumors. Genes whose overexpression has been linked to APC activation are shown in red and genes whose overexpression has been linked to T cell activation in green. FIG. 3G shows Gene Ontology (GO) analysis of pathways enriched in CD45+ fractions from LLC-Vkine versus LLC-EV tumors. FIG. 3H shows expression pattern of murine Irf8, Batf3 and Id2. Data from BioGPS. FIG. 3I shows an exemplary schematic layout of the pre-DC adoptive transfer experiment. Pre-DC were harvested from the BM of Flt3l-in vivo mobilized CD45.2+ mice and adoptively transferred into LLCEV or LLC-Vkine tumors implanted in CD45.1+ recipients. In vivo pre-DC mobilization was achieved through implantation of Flt3l-secreting B16 cells, according to standard protocols. 72 hours post-adoptive transfer, tumors were harvested, processed and cDC subsets were analyzed by flow cytometry. FIG. 3J shows gating strategy for flow sorting pre-DC from BM of Flt3l-in vivo mobilized donors, per the schema of van Ginderachter. FIG. 3K shows representative flow plots of CD45.1+ endogenous cDC subsets (left) and frequencies (right). FIG. 3L shows representative flow plots of CD45.2+ adoptively-transferred cDC subsets (left) and frequencies (right). FIG. 3M shows summary of CD40 staining intensity (MFI=mean fluorescence intensity) in DC subsets from LLC-EV and LLC-Vkine tumors.

FIGS. 4A-4K show cDC1 activation by versikine is cell autonomous. FIG. 4A shows an exemplary schematic layout of the experiment. MutuDC1940-EV or -Vkine cells were stimulated for 4 h with vehicle (PBS) or the TLR4 agonist lipopolysaccharide (LPS) (100 ng/mL) before RNA extraction. FIG. 4B shows gross morphology of MutuDC1940 cells engineered to express versikine (Vkine) or empty vector (EV). Phase contrast, 1003magnification; scale bar, 220 mm. FIG. 4C shows hierarchical clustering of MutuDC1940 transcriptomic profiles expressing EV or versikine (Vkine) and stimulated with the TLR4 agonist LPS or vehicle (PBS). FIG. 4D shows volcano plot highlighting key differentially expressed genes in MutuDC1940-Vkine versus −EV cells (without LPS). FIG. 4E shows gene set enrichment analysis (GSEA) of significantly upregulated (left and center) and downregulated (right) pathways in MutuDC1940-Vkine versus −EV cells (without LPS). FIG. 4F shows Ccl7 RT-PCR using LLC-EV and LLC-Vkine tumor bulk mRNA (left) and CD11c+ magnetically separated fraction mRNA (right). FIG. 4G shows Cxcl9 and Cxcl10 RT-PCR using CD11c+ magnetically separated fractions from LLC-EV or LLC-Vkine tumors. FIG. 4H shows and exemplary schematic of the antigen presentation experiment. FIG. 4I shows flow cytometry for endogenous IFN-g and IL-2 of OT-I CD8+ T cells co-cultured with SIINFEKL peptide-loaded MutuDC1940 cells, EV- or Ykine-expressing, with or without LPS. FIG. 4J shows quantitation of OT-I flow cytometry analysis of the antigen presentation assay. FIG. 4K shows IFN-g by ELISA in supernatants from OT-I and MutuDC1940:SIINFEKL co-cultures in the antigen presentation assay. FIG. 4L shows gene ontology (GO) pathway analysis of differentially expressed genes between MutuDC1940-Vkine vs. −EV. Versikine's proapoptotic program (“positive regulation of programmed cell death/“positive regulation of apoptotic process”) is reminiscent of versikine's proapoptotic activities during development. FIG. 4M shows RT-PCR of Cxcl9/10 in MutuDC1940-EV vs. MutuDC1940-Vkine stimulated with TLR4 agonist LPS or vehicle (PBS). FIG. 4N shows ELISA detection of secreted Cxcl9 by MutuDC1940-EV- and MutuDC1940-Vkine stimulated with LPS or vehicle (PBS) plotted against time (hours). FIG. 4O shows RT-PCR for Il27p28 and Ebi3 message in MutuDC1940-EV-vs. −Vkine stimulated with LPS or vehicle (PBS). FIG. 4P shows ELISA detection of secreted Il27p28 by MutuDC1940-EV- and MutuDC1940-Vkine stimulated with LPS or vehicle (PBS) plotted against time (hours). FIG. 4Q shows RT-PCR for selected versikine-signature genes using RNA from MutuDC1940 cells (unmanipulated) exposed to supernatant from versikine-secreting HEK293 cells (Vkine sup) vs. control supernatant (control sup) at 72 hours. FIG. 4R shows flow cytometry for endogenous IFNg and IL2 of OT-I CD8+ T cells at baseline (left) and PMA-stimulated, prior to addition of DC (right). FIG. 4S shows FMO controls shown for IFNg (left) and IL2 (right) under conditions of maximal stimulation (Vkine+LPS, compare to FIG. 4I). FIG. 4T shows IFNγ by ELISA in supernatants from OT-I+ MutuDC1940:SIINFEKL co-cultures in the antigen presentation assay.

FIGS. 5A-5G show cDC1 accumulation requires innate lymphoid support. FIG. 5A shows RT-PCR for NK cell-activating cytokine transcripts expressed by ex vivo magnetically separated CD11c+ cells from LLC-EV and LLC-Vkine tumors. FIG. 5B shows RT-PCR profile of NKp46+NK1.1+ cells flow-sorted from LLC-EV and LLC-Vkine tumors. FIG. 5C shows an exemplary schematic of the NK cell depletion experiment. FIG. 5D shows summary of cDC subset frequency by flow cytometric analysis in LLC-EV versus LLC-Vkine tumors after treatment with NK cell-depleting antibody (anti-ASGM1) or vehicle (PBS). FIG. 5E shows Csf2 (GM-CSF) RT-PCR of RNA extracted from NKp46+NK1.1+ cells flow-sorted from LLC-EV and LLC-Vkine tumors growing in WT or Batf3_/_hosts. FIG. 5F shows stromal localization of NCR1+(NKp46+) cells inhuman lung cancers (chromogen. DAB; counterstain, hematoxylin). FIG. 5G shows annexin V/7-AAD apoptosis assay of MutuDC1940-EV or -Vkine dendritic cells exposed to graded staurosporine concentrations with or without murine GMCSF. FIG. 5H shows validation of intratumoral NK (NK1.1+CD49b+) depletion following anti-ASGM1 treatment. FIG. 5I shows flow cytometric analysis of cDC subsets in LLC-EV vs. LLC-Vkine tumors following treatment with NK-depleting antibody (anti-ASGM1) or vehicle (PBS). FIG. 5J shows flow cytometric analysis of intratumoral basophils (defined as CD45intCD49b+FcεRI+IgE+c-Kit⁻ cells, gating per. FIG. 5K shows absolute counts/mg tumor tissue of intratumoral basophils compared to intratumoral NK1.1+CD3− cells, in LLC-EV vs. LLC-Vkine tumors. FIG. 5L shows absence of intratumoral cDC1 in Batf3−/− recipients by multiparametric flow cytometry. FIG. 5M shows flow cytometric analysis of cDC subsets in LLC-EV vs. LLC-Vkine tumors implanted in WT or Tlr2−/− recipients. FIG. 5N shows summary of cDC subset frequency by flow cytometric analysis in LLC-EV vs. LLC-Vkine tumors implanted in WT or Tlr2−/− recipients. FIG. 5O shows growth rates of LLC-EV and LLC-Vkine tumors in WT vs. Tlr2−/− background. FIG. 5P shows flow cytometric analysis of cDC subsets in LLC-EV vs. LLC-Vkine tumors implanted in WT or Cd44−/− recipients. FIG. SQ shows summary of cDC subset frequency by flow cytometric analysis in LLC-EV vs. LLC-Vkine tumors implanted in WT or Cd44−/− recipients. FIG. 5R shows growth rates of LLC-EV and LLC-Vkine tumors in WT vs. Cd44−/− genetic background.

FIGS. 6A-6L show stroma-licensed cDC1s are “poised” and hypersensitive to nucleic acid sensing in vivo. FIG. 6A shows an exemplary schematic of the experiment. FIG. 6B shows growth curves of LLC-EV and LLC-Vkine tumors challenged with a single subtherapeutic dose (200 mg) of intratumoral (IT) DMXAA (DMXAA200) or vehicle (NaHCO₃) on day 0. FIG. 6C shows Kaplan-Meier survival curves for the experiment in (B); **p<0.01 by log rank test. FIG. 6D shows representative images showing development of hemorrhagic necrosis and a necrotic eschar in LLC-Vkine but not LLC-EV tumors 24 h after IT DMXAA200 administration. FIG. 6E shows transcriptomic profile of LLC-EV and LLC-Vkine tumors harvested 2 h after IT DMXAA200. FIG. 6F shows versikine −DMXAA synergy generates an abscopal effect in LLC tumors that produces a survival advantage. **p<0.01 by log rank test. FIG. 6G shows growth curves of treatment-side LLC-EV and LLC-Vkine tumors challenged with a single subtherapeutic dose (200 mg) of IT DMXAA (DMXAA200) or vehicle (NaHCO₃) on day 0. FIG. 6H shows growth curves of contralateral side unmanipulated LLC tumors; treated side as in FIG. 6G. FIG. 6I shows response to DMXAA200 is lost in Batf3^{-/-} recipients. Shown are growth curves of LLC-EV and LLC-Vkine tumors challenged with a single subtherapeutic dose (200 mg) of IT DMXAA (DMXAA200) or vehicle (NaHCO3) on day 0 in Batf3^{-/-} recipients. FIG. 6J shows Batf3 loss abrogates the survival advantage seen in the WT (FIG. 6C). FIG. 6K shows efficacy of DMXAA200 in LLC-Vkine tumors implanted into Batf3^{-/-} recipients is restored after adoptive transfer of iCD103. FIG. 6L shows adoptive transfer of iCD103 in LLC-Vkine tumors implanted into Batf3^{-/-} recipients restores the survival advantage of mice treated with DMXAA200. FIG. 6M shows Versikine-DMXAA synergy generates an abscopal effect in 4T1 mammary carcinomas. Growth curves of treatment-side 4T1-EV and 4T1-Vkine tumors challenged with a single sub-therapeutic dose (200 mcg) of IT DMXAA on Day 0 (DMXAA200) or vehicle (NaHCO₃). FIG. 6N shows growth curves of contralateral side unmanipulated 4T1 tumors, according to corresponding treatment side configuration (treatment as in FIG. 6M). FIG. 6O shows Versikine-induced abscopal effect is accompanied by a survival advantage in 4T1 tumors. **=p<0.01 by log-rank test. FIG. 6P shows an exemplary schematic layout of iCD103 cell adoptive transfer experiments. FIG. 6Q shows flow-cytometric validation of the iCD103 cells, generated as described in the protocol by Merad, Sparwasser and colleagues, using standard cDC1 markers. FIG. 6R shows growth curves of B16-EV and B16-Vkine tumors challenged with a single subtherapeutic dose (200 mcg) of IT DMXAA on Day 0 (DMXAA200) or vehicle (NaHCO₃). FIG. 6S shows Kaplan-Meier survival curves for the experiment in panel S6F, *=p<0.05 by log-rank test. FIG. 6T shows response to DMXAA200 is lost in B16-Vkine tumors implanted in Batf3−/− recipients. FIG. 6U shows efficacy of sub-therapeutic DMXAA200 in B16-Vkine tumors implanted in Batf3−/− recipients is restored following adoptive transfer of iCD103 cells. A subset of B16-bearing tumors did not “take” iCD103 cells, likely attributable to the pauci-immune environment of B16 tumors.

FIGS. 7A-7F show versikine promotes CD8+ responses and overcomes resistance to anti-PD1 inhibitors in vivo. FIG. 7A shows an exemplary schematic of the experiment. FIG. 7B shows frequency of MHCI:SIINFEKL tetramer+ CD8+ splenocytes in mice bearing LLC-EV versus LLC-Vkine tumors 5 days after challenge with a therapeutic dose of a STING agonist (DMXAA500). FIG. 7C shows correlation between in vitro versikine signature and CD8+ T cell scores across TCGA human lung cancers. Significance was measured using a linear model while accounting for total immune infiltration. FIG. 7D shows DPEAAE staining in human lung cancers and associated CD8+ infiltration. FIG. 7E shows distribution of DPEAAE stromal staining intensity across lung cancer prognostic subgroups (pauci-immune [poor prognosis] and immune-rich [favorable prognosis] at cutoff 3 CD8+ TILs/HPF). p<0.001 by two-tailed Mann-Whitney test. FIG. 7F shows Top: schematic of the experiment. Bottom: tumor growth rates and survival curves of LLC-EV and LLC-Vkine-bearing animals treated with 3 doses of anti-PD1 antibody or isotype control. FIG. 7G shows CD8+ T cell subset frequency in the spleen of mice treated as in the schema depicted in FIG. 7A. Naïve (CD44−CD62L+), central memory [CM, (CD44+CD62L+)], effector/effector memory [E/EM, (CD44+CD62L−)]. FIG. 7H shows correlation between in vitro versikine response signature and corrected CD8+ T cell scores across TCGA lung cancers. CD8+ T cell scores corrected for immune infiltration to remove variation associated with immune state. FIG. 7I shows distribution of DPEAAE stromal staining intensity across lung cancer prognostic subgroups [pauciimmune (poor prognosis) and immune-rich (favorable prognosis) at cutoff 5 CD8+ TIL/HPF]. Low DPEAAE staining intensities are more prevalent in the pauci-immune subgroup than the immune-rich subgroup, p<0.001 by two-tailed Mann-Whitney test. FIG. 7J shows growth response curves of individual tumors in the anti-PD1 experiment depicted in FIG. 7F.

FIG. 8 shows an exemplary schematic of stromal remodeling regulating dendritic cell abundance and activity in the tumor microenvironment.

### DETAILED DESCRIPTION

T-cell-inflamed tumor microenvironments are a prerequisite for immunotherapy efficacy. However, why some tumors are inflamed, and others are not remains poorly understood. Tumor antigen cross-presentation and CD8+ T cell effector priming by stimulatory type 1 conventional dendritic cells (cDC1s) is integral to spontaneous and therapeutic anti-tumor immunity. In addition to effector priming in the lymph node and in the tumor microenvironment (TME), cDC1s regulate effector cell influx into the TME. From a translational perspective, cDC1s are crucial for responses to vaccination strategies, immune checkpoint inhibitors, and engineered immune effector cells (e.g., chimeric antigen receptor T [CAR-T]cells). Several studies have shown that stimulatory cDC1s are excluded from interdigitating tumor nestlets and locate in peritumoral stroma. However, the mechanisms that retain cDC1s at the tumor periphery remain poorly understood.

The large aggregating extracellular matrix proteoglycan versican (VCAN) is a central component of the embryonic provisional matrix, playing key non-redundant roles in development of the cardiovascular system and limbs. For example, Vcan-null mice die in utero by embryonic day 10.5 because of defects along the anterior-posterior cardiac axis. VCAN proteolysis by ADAMTS (a disintegrin and metalloproteinase with thrombospondin motifs) proteases at the Glu441-Ala442 bond (V1 isoform enumeration) is an essential requirement that acts in part through the specific neoactivities of the released bioactive N-terminal fragment (matrikine) versikine, wherein disruption of the Glu441-Ala442 proteolytic site that generates versikine leads to developmental abnormalities. Furthermore, it has been shown that VCAN proteolysis at the versikine C-terminal Glu441-Ala442 bond correlates with CD8+ T cell infiltration in solid and hematopoietic human tumors.

The present disclosure describes methods for increasing T cell activation in a tumor microenvironment (TME) in a subject that includes administering a proteolytic fragment of an extracellular matrix (ECM) proteoglycan. Also provided herein are methods for sensitizing a response to an immunotherapy in a subject in need thereof that includes administering a proteolytic fragment of an extracellular matrix (ECM) proteoglycan. Also provided herein are methods of predicting a subject's response to an immunotherapy that include (a) analyzing a biological sample obtained from the subject; and (b) detecting a proteolytic fragment of an extracellular matrix (ECM) proteoglycan in the biological sample, thereby predicting the subject's response to the immunotherapy.

Various non-limiting aspects of these methods are described herein and can be used in any combination without limitation. Additional aspects of various components of the methods described herein are known in the art.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 8, 17%, 16%, 15%, 14%, 13%, 12%, 11%, 10%, 9%, 8%, 7%, 6%, 5%, 4%, 3%, 2%, 1%, or less of the referred value.

As used herein, “biological sample” can refer to a sample generally including cells and/or other biological material. A biological sample can be obtained from a eukaryote, such as a patient derived organoid (PDO) or patient derived xenograft (PDX). Biological samples can be derived from a homogeneous culture or population of organisms or alternatively from a collection of several different organisms, for example, in a community or ecosystem.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As used herein, the terms “cancer”, “tumor”, and “carcinoma” refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a metastatic solid tumor. In some embodiments, a relevant cancer may be characterized by a hematologic tumor. In general, examples of different types of cancers known in the art include, for example, a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma stomach cancer, thyroid cancer, pancreatic cancer, renal cell carcinoma, glioblastoma and prostate cancer. In some embodiments, hematopoietic cancers can include leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, precancerous pathology such as myelodysplastic syndromes, acquired aplastic anemia, Fanconi anemia, paroxysmal nocturnal hemoglobinuria (PNH) and 5q-syndrome and the like.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelle biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, “nucleic acid” is used to include any compound and/or substance that comprise a polymer of nucleotides. In some embodiments, a polymer of nucleotides is referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

As used herein, the term “subject” refers an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Versikine, Versikine Fusions, and Versikine Combinations

As used herein, a “proteoglycan” refers a family of charged molecules containing a core protein and one or more covalently attached glycosaminoglycan side chains. Proteoglycans are important components of extracellular matrices (ECM) and have multiple functions that depend on both their protein and carbohydrate constituents. In some embodiments, the ECM proteoglycan is versican (VCAN). Versican is a large extracellular matrix proteoglycan that is present in a variety of tissues and plays key roles in multiple facets of cancer development, ranging from proliferative signaling, evasion of growth-suppressor pathways, regulation of cell death, promotion of neoangiogenesis, and tissue invasion and metastasis. Multiple lines of evidence implicate versican and its bioactive proteolytic fragments (matrikines) in the regulation of cancer inflammation and antitumor immune responses.

Matrikines can be defined as ECM-derived fragments that regulate cell activity, often in a manner distinct from that of their parent macromolecule. Proteolytic processing of versican at Glu441-Ala442 generates a bioactive N-terminal matrikine, versikine. In some embodiments, the proteolytic fragment of the ECM proteoglycan is versikine. In some embodiments, the versikine is generated by proteolysis of VCAN at the Glu441-Ala442 bond.

Some compositions of the disclosure contain a versikine amino acid sequence. In some embodiments, the versikine amino acid sequence is the sequence of SEQ ID NO: 1. In some embodiments, the versikine amino acid comprises a sequence that is at least 90% identical (e.g., at least 91%, at least 92%, at least 93%, at least 94%, at least 95%, at least 96%, at least 97%, at least 98%, at least 99%) to SEQ ID NO: 1.

SEQ ID NO: 1 - Versikine amino acid sequence
LHKVKVGKSPPVRGSLSGKVSLPCHFSTMPTLPPSYNTSEFLRIKWSKIE
VDKNGKDLKETTVLVAQNGNIKIGQDYKGRVSVPTHPEAVGDASLTVVKL
LASDAGLYRCDVMYGIEDTQDTVSLTVDGVVFHYRAATSRYTLNFEAAQK
ACLDVGAVIATPEQLFAAYEDGFEQCDAGWLADQTVRYPIRAPRVGCYGD
KMGKAGVRTYGFRSPQETYDVYCYVDHLDGDVFHLTVPSKFTFEEAAKEC
ENQDARLATVGELQAAWRNGFDQCDYGWLSDASVRHPVTVARAQCGGGLL
GVRTLYRFENQTGFPPPDSRFDAYCFKRRMSDLSVIGHPIDSESKEDEPC
SEETDPVHDLMAEILPEFPDIEIDLYHSEENEEEEEECANATDVTTTPSV
QYINGKHLVTTVPKDPEAAE

In some embodiments, the versikine is a nucleic acid molecule. In some embodiments, the versikine comprises a nucleic acid sequence of SEQ ID NO: 23. In some embodiments, the versikine nucleic acid is at least 90% identical (e.g., at least 910%, at least 92%, at least 93%, at least 94%, at least 95%, at least 96%, at least 97%, at least 98%, at least 99%) to SEQ ID NO: 23).

SEQ ID NO: 23 - Versikine nucleic acid sequence
ATGTTCATAAATATAAAGAGCATCTTATGGATGTGTTCAACCTTAATAGT
AACCCATGCGCTACATAAAGTCAAAGTGGGAAAAAGCCCACCGGTGAGGG
GCTCCCTCTCTGGAAAAGTCAGCCTACCTTGTCATTTTTCAACGATGCCT
ACTTTGCCACCCAGTTACAACACCAGTGAATTTCTCCGCATCAAATGGTC
TAAGATTGAAGTGGACAAAAATGGAAAAGATTTGAAAGAGACTACTGTCC
TTGTGGCCCAAAATGGAAATATCAAGATTGGTCAGGACTACAAAGGGAGA
GTGTCTGTGCCCACACATCCCGAGGCTGTGGGCGATGCCTCCCTCACTGT
GGTCAAGCTGCTGGCAAGTGATGCGGGTCTTTACCGCTGTGACGTCATGT
ACGGGATTGAAGACACACAAGACACGGTGTCACTGACTGTGGATGGGGTT
GTGTTTCACTACAGGGCGGCAACCAGCAGGTACACACTGAATTTTGAGGC
TGCTCAGAAGGCTTGTTTGGACGTTGGGGCAGTCATAGCAACTCCAGAGC
AGCTCTTTGCTGCCTATGAAGATGGATTTGAGCAGTGTGACGCAGGCTGG
CTGGCTGATCAGACTGTCAGATATCCCATCCGGGCTCCCAGAGTAGGCTG
TTATGGAGATAAGATGGGAAAGGCAGGAGTCAGGACTTATGGATTCCGTT
CTCCCCAGGAAACTTACGATGTGTATTGTTATGTGGATCATCTGGATGGT
GATGTGTTCCACCTCACTGTCCCCAGTAAATTCACCTTCGAGGAGGCTGC
AAAAGAGTGTGAAAACCAGGATGCCAGGCTGGCAACAGTGGGGGAACTCC
AGGCGGCATGGAGGAACGGCTTTGACCAGTGCGATTACGGGTGGCTGTCG
GATGCCAGCGTGCGCCACCCTGTGACTGTGGCCAGGGCCCAGTGTGGAGG
TGGTCTACTTGGGGTGAGAACCCTGTATCGTTTTGAGAACCAGACAGGCT
TCCCTCCCCCTGATAGCAGATTTGATGCCTACTGCTTTAAACGTCGAATG
AGTGATTTGAGTGTAATTGGTCATCCAATAGATTCAGAATCTAAAGAAGA
TGAACCTTGTAGTGAAGAAACAGATCCAGTGCATGATCTAATGGCTGAAA
TTTTACCTGAATTCCCTGACATAATTGAAATAGACCTATACCACAGTGAA
GAAAATGAAGAAGAAGAAGAAGAGTGTGCAAATGCTACTGATGTGACAAC
CACCCCATCTGTGCAGTACATAAATGGGAAGCATCTCGTTACCACTGTGC
CCAAGGACCCAGAAGCTGCAGAA

In some embodiments, the versikine nucleic acid molecule is fused to a non-versikine nucleic acid molecule. In some embodiments, the versikine nucleic acid molecule is fused to a non-versikine nucleic acid molecule via a linker. In some embodiments, the non-versikine nucleic acid molecule encodes a secretory peptide. In some embodiments, the non-versikine nucleic acid molecule encodes an immunomodulator. In some embodiments, the non-versikine nucleic acid molecule is a DNA or RNA molecule.

In some embodiments, the versikine protein is conjugated to another protein. In some embodiments, the versikine protein is conjugated to another protein via a linker. In some embodiments, the linker comprises the amino acid sequence MFINIKSILWMCSTLIVTHA (SEQ ID NO: 22). In some embodiments, the linker comprises the amino acid sequence GGGS (SEQ ID NO: 24), GGGSGGGSGGGS (SEQ ID NO: 25), GGGGGGGG (SEQ ID NO: 26), EAAAKEAAAKEAAAK (SEQ ID NO: 27), PAPAP (SEQ ID NO: 28), or AEAAAKEAAAKA (SEQ ID NO: 29). In some embodiments, the linker comprises the amino acid sequence KESGSVSSEQLAQFRSLD (SEQ ID NO: 30), EGKSSGSGSESKST (SEQ ID NO: 31), or GSAGSAAGSGEF (SEQ ID NO: 32). Additional examples of linkers are described for example in Chen et al., Adv Drug Deliv Rev. 2013 October; 65(10):1357-69, the entire contents of which are incorporated herein by reference. In some embodiments, the versikine protein is conjugated to another protein at the N-terminal. In some embodiments, the versikine protein is conjugated to another protein at the C-terminal.

### Secretory Peptides

In some embodiments, the other protein comprises a secretory peptide. In some embodiments, the versikine protein is conjugated to the secretory peptide via a linker. In some embodiments, the versikine protein is conjugated to the secretory peptide at the N-terminal. In some embodiments, the secretory peptide is selected from the group consisting of: human OSM, human immunoglobulin, human chymotrypsinogen, human trypsinogen 2, human IL-2, or human insulin.

In some embodiments, the versikine protein conjugated with the secretory peptide comprises SEQ ID NO: 1 and SEQ ID NO: 2, wherein SEQ ID NO: 2 is fused to the N terminal of SEQ ID NO: 1 or wherein SEQ ID NO: 2 is fused to the C terminal of SEQ ID NO: 1, optionally including a linker. In some embodiments, the versikine protein conjugated with the secretory peptide comprises SEQ ID NO: 1 and SEQ ID NO: 3, wherein SEQ ID NO: 3 is fused to the N terminal of SEQ ID NO: 1 or wherein SEQ ID NO: 3 is fused to the C terminal of SEQ ID NO: 1, optionally including a linker. In some embodiments, the versikine protein conjugated with the secretory peptide comprises SEQ ID NO: 1 and SEQ ID NO: 4, wherein SEQ ID NO: 4 is fused to the N terminal of SEQ ID NO: 1 or wherein SEQ ID NO: 4 is fused to the C terminal of SEQ ID NO: 1, optionally including a linker. In some embodiments, the versikine protein conjugated with the secretory peptide comprises SEQ ID NO: 1 and SEQ ID NO: 5, wherein SEQ ID NO: 5 is fused to the N terminal of SEQ ID NO: 1 or wherein SEQ ID NO: 5 is fused to the C terminal of SEQ ID NO: 1, optionally including a linker. In some embodiments, the versikine protein conjugated with the secretory peptide comprises SEQ ID NO: 1 and SEQ ID NO: 6, wherein SEQ ID NO: 6 is fused to the N terminal of SEQ ID NO: 1 or wherein SEQ ID NO: 6 is fused to the C terminal of SEQ ID NO: 1, optionally including a linker. In some embodiments, the versikine protein conjugated with the secretory peptide comprises SEQ ID NO: 1 and SEQ ID NO: 7, wherein SEQ ID NO: 7 is fused to the N terminal of SEQ ID NO: 1 or wherein SEQ ID NO: 7 is fused to the C terminal of SEQ ID NO: 1, optionally including a linker.

SEQ ID NO: 2 - Human OSM
MGVLLTQRTLLSLVLALLFPSMASM
SEQ ID NO: 3 - Human immunoglobulin
MDMRVPAQLLGLLLLWLRGARC
SEQ ID NO: 4 - Human chymotrypsinogen
MAFLWLLSCWALLGTTFG
SEQ ID NO: 5 - Human trypsinogen 2
MNLLLILTFVAAAVA
SEQ ID NO: 6 - Human IL-2
MYRMQLLSCIALSLALVTNS
SEQ ID NO: 7 - Human insulin
MALWMRLLPLLALLALWGPDPAAA

### Immunomodulators

In some embodiments, the other protein comprises an immunomodulator. In some embodiments, the versikine protein is conjugated to the immunomodulator via a linker. In some embodiments, the versikine protein is conjugated to the immunomodulator at the N-terminal. In some embodiments, the versikine protein is conjugated to the immunomodulator at the C-terminal. In some embodiments, the other protein is selected from the group consisting of: GM-CSF, CCL5, XCL1, FLT3L, CCL7, CXCL9, CXCL10, IL-12, IL-27, IL-15, IL18, IL-23, IL-2, IFNβ1, IFNα2, or IFNγ. In some embodiments, the other protein can comprise an IFN-α subtype (e.g., IFN-α1, IFN-α2, IFN-α4, IFN-α5, IFN-α6, IFN-α7, IFN-α8, IFN-α10, IFN-α13, IFN-α14, IFN-α17, or IFN-α21).

In some embodiments, the versikine protein conjugated with the immunomodulator comprises SEQ ID NO: 1 and SEQ ID NO: 8, wherein SEQ ID NO: 8 is fused to the N terminal of SEQ ID NO: 1 or wherein SEQ ID NO: 8 is fused to the C terminal of SEQ ID NO: 1, optionally including a linker. In some embodiments, the versikine protein conjugated with the immunomodulator comprises SEQ ID NO: 1 and SEQ ID NO: 9, wherein SEQ ID NO: 9 is fused to the N terminal of SEQ ID NO: 1 or wherein SEQ ID NO: 9 is fused to the C terminal of SEQ ID NO: 1, optionally including a linker. In some embodiments, the versikine protein conjugated with the immunomodulator comprises SEQ ID NO: 1 and SEQ ID NO: 10, wherein SEQ ID NO: 10 is fused to the N terminal of SEQ ID NO: 1 or wherein SEQ ID NO: 10 is fused to the C terminal of SEQ ID NO: 1, optionally including a linker. In some embodiments, the versikine protein conjugated with the immunomodulator comprises SEQ ID NO: 1 and SEQ ID NO: 11, wherein SEQ ID NO: 11 is fused to the N terminal of SEQ ID NO: 1 or wherein SEQ ID NO: 11 is fused to the C terminal of SEQ ID NO: 1, optionally including a linker. In some embodiments, the versikine protein conjugated with the immunomodulator comprises SEQ ID NO: 1 and SEQ ID NO: 12, wherein SEQ ID NO: 12 is fused to the N terminal of SEQ ID NO: 1 or wherein SEQ ID NO: 12 is fused to the C terminal of SEQ ID NO: 1, optionally including a linker. In some embodiments, the versikine protein conjugated with the immunomodulator comprises SEQ ID NO: 1 and SEQ ID NO: 13, wherein SEQ ID NO: 13 is fused to the N terminal of SEQ ID NO: 1 or wherein SEQ ID NO: 13 is fused to the C terminal of SEQ ID NO: 1, optionally including a linker. In some embodiments, the versikine protein conjugated with the immunomodulator comprises SEQ ID NO: 1 and SEQ ID NO: 14, wherein SEQ ID NO: 14 is fused to the N terminal of SEQ ID NO: 1 or wherein SEQ ID NO: 14 is fused to the C terminal of SEQ ID NO: 1, optionally including a linker. In some embodiments, the versikine protein conjugated with the immunomodulator comprises SEQ ID NO: 1 and SEQ ID NO: 15, wherein SEQ ID NO: 15 is fused to the N terminal of SEQ ID NO: 1 or wherein SEQ ID NO: 15 is fused to the C terminal of SEQ ID NO: 1, optionally including a linker. In some embodiments, the versikine protein conjugated with the immunomodulator comprises SEQ ID NO: 1 and SEQ ID NO: 16, wherein SEQ ID NO: 16 is fused to the N terminal of SEQ ID NO: 1 or wherein SEQ ID NO: 16 is fused to the C terminal of SEQ ID NO: 1, optionally including a linker. In some embodiments, the versikine protein conjugated with the immunomodulator comprises SEQ ID NO: 1 and SEQ ID NO: 17, wherein SEQ ID NO: 17 is fused to the N terminal of SEQ ID NO: 1 or wherein SEQ ID NO: 17 is fused to the C terminal of SEQ ID NO: 1, optionally including a linker. In some embodiments, the versikine protein conjugated with the immunomodulator comprises SEQ ID NO: 1 and SEQ ID NO: 18 wherein SEQ ID NO: 18 is fused to the N terminal of SEQ ID NO: 1 or wherein SEQ ID NO: 18 is fused to the C terminal of SEQ ID NO: 1, optionally including a linker. In some embodiments, the versikine protein conjugated with the immunomodulator comprises SEQ ID NO: 1 and SEQ ID NO: 19, wherein SEQ ID NO: 19 is fused to the N terminal of SEQ ID NO: 1 or wherein SEQ ID NO: 19 is fused to the C terminal of SEQ ID NO: 1, optionally including a linker. In some embodiments, the versikine protein conjugated with the immunomodulator comprises SEQ ID NO: 1 and SEQ ID NO: 20, wherein SEQ ID NO: 20 is fused to the N terminal of SEQ ID NO: 1 or wherein SEQ ID NO: 20 is fused to the C terminal of SEQ ID NO: 1, optionally including a linker. In some embodiments, the versikine protein conjugated with the immunomodulator comprises SEQ ID NO: 1 and SEQ ID NO: 21, wherein SEQ ID NO: 21 is fused to the N terminal of SEQ ID NO: 1 or wherein SEQ ID NO: 21 is fused to the C terminal of SEQ ID NO: 1, optionally including a linker. In some embodiments, the versikine protein conjugated with the immunomodulator comprises SEQ ID NO: 1 and SEQ ID NO: 33, wherein SEQ ID NO: 33 is fused to the N terminal of SEQ ID NO: 1 or wherein SEQ ID NO: 33 is fused to the C terminal of SEQ ID NO: 1, optionally including a linker. In some embodiments, the versikine protein conjugated with the immunomodulator comprises SEQ ID NO: 1 and SEQ ID NO: 34, wherein SEQ ID NO: 34 is fused to the N terminal of SEQ ID NO: 1 or wherein SEQ ID NO: 34 is fused to the C terminal of SEQ ID NO: 1, optionally including a linker.

SEQ ID NO: 8 - Human GM-CSF
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQE
SEQ ID NO: 9 - Human CCL5
MKVSAAALAVILIATALCAPASAFPYSSDTTPCCFAYIARPLPRAHIKEY
FYTSGKCSNPAVVFVTRKNRQVCANPEKKWVREYINSLEMS
SEQ ID NO: 10 - Human XCL1
MRLLILALLGICSLTAYIVEGVGSEVSDKRTCVSLTTQRLPVSRIKTYTI
TEGSLRAVIFITKRGLKVCADPQATWVRDVVRSMDRKSNTRNNMIQTKPT
GTQQSTNTAVTLTG
SEQ ID NO: 11 - Human FLT3LG
MTVLAPAWSPTTYLLLLLLLSSGLSGTQDCSFQHSPISSDFAVKIRELSD
YLLQDYPVTVASNLQDEELCGGLWRLVLAQRWMERLKTVAGSKMQGLLER
VNTEIHFVTKCAFQPPPSCLRFVQTNISRLLQETSEQLVALKPWITRQNF
SRCLELQCQP
DSSTLPPPWSPRPLEATAPTAPQPPLLLLLLLPVGLLLLAAAWCLHWQRT
RRRTPRPGEQVPPVPSPQDLLLVEH
SEQ ID NO: 12 - Human CCL7
MKASAALLCLLLTAAAFSPQGLAQPVGINTSTTCCYRFINKKIPKQRLES
YRRTTSSHCPREAVIFKTKLDKEICADPTQKWVQDFMKHLDKKTQTPKL
SEQ ID NO: 13 - Human CXCL9
MKKSGVLFLLGIILLVLIGVQGTPVVRKGRCSCISTNQGTIHLQSLKDLK
QFAPSPSCEKIEIIATLKNGVQTCLNPDSADVKELIKKWEKQVSQKKKQK
NGKKHQKKKVLKVRKSQRSRQKKTT
SEQ ID NO: 14 - Human CXCL 10
MNQTAILICCLIFLTLSGIQGVPLSRTVRCTCISISNQPVNPRSLEKLEI
IPASQFCPRVEIIATMKKKGEKRCLNPESKAIKNLLKAVSKERSKRSP
SEQ ID NO: 15 - Human IL-12
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCS
SEQ ID NO: 16 - Human IL-27
MGQTAGDLGWRLSLLLLPLLLVQAGVWGFPRPPGRPQLSLQELRREFTVS
LHLARKLLSEVRGQAHRFAESHLPGVNLYLLPLGEQLPDVSLTFQAWRRL
SDPERLCFISTTLQPFHALLGGLGTQGRWTNMERMQLWAMRLDLRDLQRH
LRFQVLAAGFNLPEEEEEEEEEEEEERKGLLPGALGSALQGPAQVSWPQL
LSTYRLLHSLELVLSRAVRELLLLSKAGHSVWPLGFPTLSPQP
SEQ ID NO: 17 - Human IL-15
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS
SEQ ID NO: 18 - Human IL-18
MAAEPVEDNCINFVAMKFIDNTLYFIAEDDENLESDYFGKLESKLSVIRN
LNDQVLFIDQGNRPLFEDMTDSDCRDNAPRTIFIISMYKDSQPRGMAVTI
SVKCEKISTLSCENKIISFKEMNPPDNIKDTKSDIIFFQRSVPGHDNKMQ
FESSSYEGYFLACEKERDLFKLILKKEDELGDRSIMFTVQNED
SEQ ID NO: 19 - Human IL-23
MLGSRAVMLLLLLPWTAQGRAVPGGSSPAWTQCQQLSQKLCTLAWSAHPL
VGHMDLREEGDEETTNDVPHIQCGDGCDPQGLRDNSQFCLQRIHQGLIFY
EKLLGSDIFTGEPSLLPDSPVGQLHASLLGLSQLLQPEGHHWETQQIPSL
SPSQPWQRLLLRFKILRSLQAFVAVAARVFAHGAATLSP
SEQ ID NO: 20 - Human IL-2
MYRMQLLSCIALSLALVINSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT
SEQ ID NO: 21 - Human IFNgamma (IFNγ)
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ
SEQ ID NO: 33 - Human IFNβ1
MTNKCLLQIALLLCFSTTALSMSYNLLGFLQRSSNFQCQKLLWQLNGRLE
YCLKDRMNFDIPEEIKQLQQFQKEDAALTIYEMLQNIFAIFRQDSSSTGW
NETIVENLLANVYHQINHLKTVLEEKLEKEDFTRGKLMSSLHLKRYYGRI
LHYLKAKEYSHCAWTIVRVEILRNFYFINRLTGYLRN
SEQ ID NO: 34 - Human IFNα2
MALTFALLVALLVLSCKSSCSVGCDLPQTHSLGSRRTLMLLAQMRRISLF
SCLKDRHDFGFPQEEFGNQFQKAETIPVLHEMIQQIFNLFSTKDSSAAWD
ETLLDKFYTELYQQLNDLEACVIQGVGVTETPLMKEDSILAVRKYFQRIT
LYLKEKKYSPCAWEVVRAEIMRSFSLSTNLQESLRSKE

The secretory peptides fused to versikine may optionally be fused to the immunomodulators of the disclosure. In some embodiments, the fusion proteins of the disclosure can be represented by the formula A-L-V-B, wherein A is selected from the group consisting of SEQ ID NOs: 2-7, 8-21, or 33-34, L comprises a linker sequence, V comprises an amino acid having at least 90% identity to SEQ ID NO: 1, and B is selected from the group consisting of SEQ ID NOs: 8-21, and 33-34.

In some embodiments, the versikine nucleic acid molecule is fused to a non-versikine nucleic acid molecule. In some embodiments, the versikine nucleic acid molecule is fused to a non-versikine nucleic acid molecule via a linker. In some embodiments, the non-versikine nucleic acid molecule encodes a secretory peptide. In some embodiments, the non-versikine nucleic acid molecule encodes an immunomodulator. In some embodiments, the non-versikine nucleic acid molecule is a DNA or RNA molecule.

In some embodiments, the versikine protein is conjugated to a drug. In some embodiments, the drug comprises a small molecule STING agonist. In some embodiments, the versikine protein is conjugated to a small molecule. In some embodiments, the versikine protein is conjugated to a radiochemical. In some embodiments, the versikine protein is conjugated to an immunogenic component. In some embodiments, the versikine protein is conjugated to a label. In some embodiments, the label is a radioisotope or a fluorophore. In some embodiments, the versikine protein is conjugated to a small molecule immunomodulator. In some embodiments, a small molecule immunomodulator comprises an indoleamine 2,3-dioxygenase 1 (IDO1) inhibitor. In some embodiments, the indoleamine 2,3-dioxygenase 1 (IDO1) inhibitor comprises Indoximod, Epacadostat, BMS-986205, Navoximod (GDC-0919, NLG-919), PF-06840003, KHK2455, or LY3381916.

Compositions Containing Versikine DNA and/or Versikine Fusion Proteins

Provided herein are compositions containing versikine DNA and/or versikine fusion proteins. In some embodiments, the compositions of the disclosure are pharmaceutical compositions comprising versikine DNA and/or versikine fusion proteins described herein. As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Provided herein are vectors comprising any one of the nucleic acids described herein. As used herein. “vector” refers to a nucleic acid molecule capable of transporting another nucleic acid to which it has been linked. One type of vector is a plasmid, which refers to a circular double stranded DNA loop into which additional DNA segments may be ligated.

Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. As used herein, the term “lentivirus” refers to a genus of the Retroviridae family. Lentiviruses are unique among the retroviruses in being able to infect non-dividing cells; they can deliver a significant amount of genetic information into the DNA of a host cell, so they are one of the most efficient methods of a gene delivery vector. HIV, SIV, and FIV are all examples of lentiviruses. Vectors derived from lentiviruses offer the means to achieve significant levels of gene transfer in vivo.

Exemplary viral vectors include polyoma, SV40, adenovirus, vaccinia virus, adeno-associated virus, herpes viruses including HSV and EBV, lentivirus, Sindbis viruses, alphaviruses and retroviruses of avian, murine, and human origin. Baculovirus (Autographa californica multinuclear polyhedrosis virus; AcMNPV) vectors can be used and obtained from commercial sources. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes simplex virus and adenoviruses, and RNA viruses such as retroviruses and polio.

Certain vectors are capable of directing the expression of genes to which they are operatively-linked. Such vectors are referred to herein as “expression vectors.” Common expression vectors are often in the form of plasmids. Recombinant expression vectors can comprise a nucleic acid provided herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual 2^{nd} ed., Cold Spring Harbor Laboratory Press, Cold Spring Harbor, N.Y. (1989)), which is incorporated herein by reference for any purpose.

### In Vitro Applications of Versikine Compositions

The term “in vitro” as used herein refers to events that occur in an artificial environment, e.g., in a test tube or reaction vessel, in cell culture, etc., rather than within a multi-cellular organism.

The veriskine nucleotides and/or the versikine pharmaceutical compositions described herein may be used in in vitro applications. In some embodiments, the administering of a proteolytic fragment of an ECM proteoglycan (e.g., veriskine) comprises delivery of the proteolytic fragment into a cell, wherein the cell is not currently integrated into a multi-cellular organism (e.g., a living subject). In some embodiments, the cell is part of a cell culture. In some embodiments, the cell is part of an organoid. In some embodiments, a cell is a primary hematopoietic, a non-hematopoietic human cell, or an iPS (inducible pluripotent stem cell). In some embodiments, the cell is an antigen-presenting cell (e.g., a macrophage, B cell, or dendritic cell). In some embodiments, the cell is a non-hematopoietic antigen-presenting cell (e.g., antigen-presenting fibroblast, lymph node stromal cell, or endothelial cell). In some embodiments, the versikine can be delivered into a cell by a variety of well-known techniques. In some embodiments, the delivery method can include a vector-mediated gene transfer (e.g., viral infection/transfection, or various other protein-based or lipid-based gene delivery complexes). In some embodiments, the delivery method can include methods facilitating the delivery of “naked” polynucleotides (e.g., electroporation, “gene gun” delivery and various other techniques used for the introduction of polynucleotides). In some embodiments, the delivering comprises transfection, electroporation, or a virus-based delivery. In some embodiments, the delivery of the proteolytic fragment comprises a virus-based delivery. In some embodiments, the virus-based delivery comprises an adeno-associated virus or a lentivirus. In some embodiments, the virus-based delivery comprises a lentivirus. In some embodiments, the versikine nucleic acid can be introduced into/uptaken by dendritic or other antigen-presenting cells (e.g., macrophage, B cell, and/or dendritic cell) to generate cell-based vaccines. For example, in some embodiments, the versikine nucleic acid can be introduced into a dendritic cell and enhancing antigen-presentation capacity of the dendritic cell, thereby generating a DC cellular vaccine. For such cell-based vaccines, cell lines are grown, and the versikine nucleic acid (SEQ ID NO: 23) is introduced to the cell via a viral vector. The virus strain replicates in the mammalian cells and is then extracted from the cells and purified. In some embodiments, a versikine nucleic acid can be stably introduced into a cell for vaccine production. In some embodiments, a versikine nucleic acid can be delivered into a cell via standard transfection or using lipid nanoparticles (LNPs). In some embodiments, a versikine protein can be expressed ex vivo and introduced into a subject.

### In Vivo Applications of Versikine Compositions

The term “in vivo” as used herein refers to events that occur within a multi-cellular organism, such as a human and a non-human animal. In the context of cell-based systems, the term may be used to refer to events that occur within a living cell (as opposed to, for example, in vitro systems).

The veriskine nucleotides and/or the versikine pharmaceutical compositions described herein may be used in in vivo applications. In some embodiments, the administering of a proteolytic fragment of an ECM proteoglycan (e.g., veriskine) comprises delivery of the proteolytic fragment into a cell, wherein the cell is integrated into a subject. In some embodiments, the cell of the subject is an antigen-presenting cell (e.g., a macrophage, B cell, or dendritic cell). In some embodiments, the cell of the subject is a non-hematopoietic antigen-presenting cell (e.g., antigen-presenting fibroblast, lymph node stromal cell, or endothelial cell). In some embodiments, the versikine can be delivered into a cell by a variety of well-known techniques. In some embodiments, the delivery method can include a vector-mediated gene transfer (e.g., viral infection/transfection, or various other protein-based or lipid-based gene delivery complexes). In some embodiments, the delivering comprises transfection or a virus-based delivery. In some embodiments, the delivery of the proteolytic fragment comprises a virus-based delivery. In some embodiments, the virus-based delivery comprises an adeno-associated virus or a lentivirus. In some embodiments, the virus-based delivery comprises a lentivirus. In some embodiments, the versikine composition administered as described herein treats cancer by increasing T cell activation in a tumor microenvironment, and/or potentiating the effects of other cancer treatment.

The compositions described herein can be used to treat cancer. The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like,

### Administration of Versikine

In some embodiments, the versikine DNA, fusion proteins, and/or the compositions described herein may be administered alone or with an immunotherapy agent and/or a pharmaceutical agent. In some embodiments, the pharmaceutical agent comprises a protein selected from the group consisting of: GM-CSF, CCL5, XCL1, FLT3L, CCL7, CXCL9, CXCL10, IL-12, IL-27, IL-15, IL18, IL-23, IL-2, or IFN. In some embodiments, the immunotherapy agent comprises a STING agonist, a PD-1 inhibitor, a PD-L1 inhibitor, or a CD40 agonist. In some embodiments, the versikine and the pharmaceutical agent can be administered simultaneously. In some embodiments, the versikine can be administered before administering the pharmaceutical agent. In some embodiments, the pharmaceutical agent can be administered after administering the versikine composition.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this disclosure would be indicated by finding a decrease in any of the pro-angiogenic markers discussed above, an increase in anti-angiogenic markers described herein, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### Immunotherapy/Methods of Treating Cancer

As used herein, “immunotherapy” refers to a treatment of disease (e.g., cancer) by activating or suppressing the immune system. For example, cancer immunotherapy uses the immune system and its components to mount an anti-tumor response through immune activation. In some embodiments, an immunotherapy can include an immune checkpoint inhibitor, an oncolytic virus therapy, a cell-based therapy, a CAR-T cell therapy, or a cancer vaccine. In some embodiments, an immunotherapy can include immune checkpoint blockade (ICB), wherein an immune checkpoint inhibitor is administered. Immune checkpoint blockade (ICB) is a treatment that uses immune checkpoint inhibitors to address a disease (e.g., cancer), wherein the immune checkpoint inhibitor blocks a checkpoint protein from binding to its partner protein or receptor.

The versikine compositions of the disclosure may be used in methods of treating cancer and/or in conjunction with other immunotherapy. In some embodiments, the immunotherapy includes administration of an immune checkpoint inhibitor. In some embodiments, the immune checkpoint inhibitor is a PD-1 inhibitor. Examples of a PD-1 inhibitor that can be used in conjunction with versikine compositions of the disclosure can include, but are not limited to, pembrolizumab, nivolumab, cemiplimab, JTX-4014, spartalizumab, camrelizumab, sintilimab, tislelizumab, toripalimab, and dostarlimab. In some embodiments, the immune checkpoint inhibitor is a PD-L1 inhibitor. Examples of a PD-L1 inhibitor can include, but are not limited to, atezolizumab, avelumab, durvalumab, KN035, CK-301, AUNP12, CA-170, and BMS-986189. In some embodiments, the immune checkpoint inhibitor can be any checkpoint inhibitor, e.g., as described in Mazzarella et al., Eur J Cancer (2019) 117:14-31, hereby incorporated by reference. In some embodiments, the immunotherapy comprises an anti-PD1 immunotherapy.

In some embodiments, the immunotherapy comprises a stimulator of interferon genes (STING) agonist immunotherapy. In some embodiments, the immunotherapy includes activation of the cGAS-STING signaling pathway (e.g., STING agonist immunotherapy). Activation of the cyclic GMP-AMP synthase (cGAS)-stimulator of interferon genes (STING) signaling pathway induces the expression of type I interferons and proinflammatory cytokines, promoting a robust adaptive antitumor immunity. As part of this innate immune signaling pathway, STING is ubiquitously expressed in immune and nonimmune cells, wherein STING activation has been demonstrated to propagate the cancer immunity cycle, remodel the tumor microenvironment, and ultimately eliminate tumor cells. Non-limiting examples of STING agonists that can be used in conjunction with versikine compositions of the disclosure include ENPP1 inhibitors (e.g., MV-626, SR-8314, SR-8291, SR8541A), bacterial vectors (e.g., SYNBT891 and STACT-TREX-1), CDN compounds (ADU-S100, BI-STING, BMS-986301, GSK532, JNJ-4412, MK-1454, SB11285, 3′3′cyclic AIMP), non-CDN small molecules (e.g., ALG-031048, ASA404, DMXAA, E7766, JNJ-′6196, MK-2118, MSA-1, MSA-2, SNX281, SR-717, TAK676, TTI-100001), nanovaccines (e.g., PC7A NP, cGAMP-NP, ONM-500), antibody-drug conjugates (e.g., XMT-2056 and CRD-5500), and exoSTING. In some examples, the STING agonist is DMXAA.

In some embodiments, the immunotherapy includes CD40 agonist immunotherapy. CD40 is a cell-surface member of the TNF (tumor necrosis factor) receptor superfamily, and upon activation, CD40 can license dendritic cells (DCs) to promote antitumor T cell activation and re-educate macrophages to destroy tumor stroma. CD40 activation can play a role in driving antitumor immunity, whereby CD40-activated DCs are poised to prime or activate tumor-specific T cells. In some embodiments. CD40 agonists can be used as an immunotherapy for patients with cancer, wherein CD40 agonist immunotherapy can include agonistic anti-CD40 monoclonal antibodies (mAbs), trimeric CD40 ligand (CD40L), or ectopic expression of CD40L using gene therapy of transferred tumor or other cells. In some embodiments, the immunotherapy comprises an anti-CD40 immunotherapy. Non-limiting examples of CD40 agonists that can be used in conjunction with versikine compositions of the disclosure include antibodies (e.g., selicrelumab (CP-870,893 or RO7009789), APX005M, JNJ-64457107, SEA-CD40, ChiLob7/4, CDX-1140H, Dacetuzumab (SGN-40), ABBV-428).

Provided herein are methods for increasing T cell activation in a tumor microenvironment (TME) in a subject that include administering a proteolytic fragment of an extracellular matrix (ECM) proteoglycan (e.g., versikine).

As used herein, “a tumor microenvironment” (TME) refers to a highly complex and dynamic structure of cells of which a variety of immune cells are a major component. The TME contains cells of the immune system (e.g., T cells, B cells, dendritic cells, myeloid-derived suppressor cells (MDSCs), tumor-associated macrophages (TAMs)), a complicated network of fibroblasts, blood vessels, lymphatics, and the cells of the cancer itself. These TME elements are surrounded by a dense meshwork of collagen and elastin fibers that comprise the extracellular matrix (ECM), wherein the TME is also composed of a complicated network of cytokines, chemokines, growth factors, and inflammatory, as well as matrix remodeling enzymes. Furthermore, the TME is a critical facilitator of immune escape and cancer progression, and the interaction between cancer cells and the diverse cell population within the TME influences tumor resistance, progression, and metastasis.

In some embodiments, the administering of a proteolytic fragment of an ECM proteoglycan (e.g., veriskine) comprises delivery of the proteolytic fragment into a cell, thereby increasing T cell activation in a tumor microenvironment. In some embodiments, the cell is an antigen-presenting cell (e.g., a macrophage, B cell, or dendritic cell). In some embodiments, the cell is a non-hematopoietic antigen-presenting cell (e.g., antigen-presenting fibroblast, lymph node stromal cell, or endothelial cell). In some embodiments, the proteolytic fragment can be delivered into a cell by a variety of well-known techniques. In some embodiments, the delivery method can include a vector-mediated gene transfer (e.g., viral infection/transfection, or various other protein-based or lipid-based gene delivery complexes). In some embodiments, the delivery method can include methods facilitating the delivery of “naked” polynucleotides (e.g., electroporation, “gene gun” delivery and various other techniques used for the introduction of polynucleotides). In some embodiments, the delivering comprises transfection, electroporation, or a virus-based delivery. In some embodiments, the delivery of the proteolytic fragment comprises a virus-based delivery. In some embodiments, the virus-based delivery comprises an adeno-associated virus or a lentivirus. In some embodiments, the virus-based delivery comprises a lentivirus. In some embodiments, the versikine composition administered as described herein increases T cell activation in a tumor microenvironment, thereby treating cancer and/or potentiating the effects of other cancer treatment.

Provided herein are methods for sensitizing a response to an immunotherapy in a subject in need thereof that include administering a proteolytic fragment of an extracellular matrix (ECM) proteoglycan (e.g., versikine) to a subject in need thereof. As used herein, the term “sensitizing” or “improve sensitivity” can refer to increasing response rates to a specific treatment (e.g., immunotherapy). In some embodiments, improving sensitivity to ICB treatment can refer to inhibiting negative regulatory immune checkpoints or stimulating activating immune checkpoints. In some embodiments, the subject has refractory cancer (e.g., a refractory tumor). In some embodiments, the subject has undergone previous treatments for cancer. In some embodiments, the subject has not shown improvement in cancer symptoms and/or cancer markers after previous treatments for cancer. In some embodiments, administration of the versikine compositions sensitizes a response to immunotherapy in a subject, such that the subject shows improvement in cancer symptoms and/or cancer markers over similar immunotherapy without administration of versikine compositions.

### Methods of Predicting a Subject's Response to an Immunotherapy

Provided herein are methods of predicting a subject's response to an immunotherapy that include (a) analyzing a biological sample obtained from the subject; and (b) detecting a proteolytic fragment of an extracellular matrix (ECM) proteoglycan in the biological sample, thereby predicting the subject's response to the immunotherapy.

In some embodiments, the analyzing comprises staining the biological sample. In some embodiments, non-limiting examples of stains can include histological stains (e.g., hematoxylin and/or eosin) and immunological stains (e.g., fluorescent stains). In some embodiments, the staining includes the use of hematoxylin and eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some embodiments, the staining can include the use of a detectable label selected from the group consisting of a radioisotope, a fluorophore, a chemiluminescent compound, a bioluminescent compound, or a combination thereof.

In some embodiments, the staining comprises an immunofluorescence stain. In some embodiments, the staining comprises an immunohistochemistry stain. In some embodiments, the biological sample is stained with an antibody. In some embodiments, the antibody is selected from the group consisting of: an anti-DPEAAE neo-epitope antibody, anti-HA antibody, anti-XCR1 antibody, anti-CLEC9A antibody, anti-NCR1 antibody, and anti-CD8 antibody.

In some embodiments, the detecting can include imaging of the biological sample (e.g., a fixed and/or stained biological sample). In some embodiments, the biological sample can be visualized or imaged using bright field microscopy. In some embodiments, the biological sample can be visualized or imaged using fluorescence microscopy. In some embodiments, non-limiting examples of visualization and imaging can include expansion microscopy, bright field microscopy, dark field microscopy, phase contrast microscopy, electron microscopy, fluorescence microscopy, reflection microscopy, interference microscopy and confocal microscopy.

In some embodiments, the detecting comprises determining the level of the proteolytic fragment by measuring the antibody in the biological sample.

### EXAMPLES

### Lentiviral Transduction

HEK293T cells were transfected with a mixture of ps-PAX2 (packaging plasmid) and pVSV-G (envelope plasmid), and transfer plasmids encoding respective open reading frames or empty control. On Day 2 post-transfection, pseudotype virus-containing culture medium was harvested, filtered, supplemented with 7.5 mg/mL polybrene (Sigma-Aldrich), and immediately applied to target cells for spinfection (120 min, 2500×g at 32 C). After spinfection, the medium was exchanged for fresh complete RPMIT640 medium. Target cells were passaged at least three times after retroviral transduction.

### Generation of HA-Tagged Versikine- and OVA-ZsGreen-Expressing Cell Lines

LLC, 4T1, B16-F10 melanoma and MutuDC1940 cells were transduced with HA tagged versikine (Vkine)—or empty vector (EV)-containing lentivirus as detailed above. The cells were selected with 10 mg/mL blasticidin for 2 weeks. HA-tagged versikine expression was confirmed by western blotting using anti-HA antibody (clone: C29F4, Cell Signaling). LLC-EV or -Vkine cell lines were transduced with pHIV-Luc-OVA-ZsGreen lentivirus. LLC-OVA expressing cells were FACS-sorted based on ZsGreen expression to ensure comparable transduction rates between different cell lines.

shRNA Mediated VCAN Knockdown

The lentiviral shRNA vector set targeting mouse Vcan (NM_019389.2) and scrambled control were purchased from GeneCopoeia (#MSH080253-LVRU6H and #CSHCTR001-LVRU6H). In brief, 2×10⁵ LLC cells were plated per well in a 6-well plate and incubated overnight. Next day, 2 mL freshly harvested lentiviral supernatant (expressing either shambled control, Vcan shRNA #1, #2 or #3), 1 mL of culture medium and 7.5 mg/mL polybrene was added per well. The plate was centrifuged at 800 g for 2 h at 37 °C and returned to CO2 incubator. After 72 h, 200 mg/mL Hygromycin B was added and the cells were under antibiotic selection for 2 weeks. Vcan knockdown was confirmed by RT-PCR.

### Tumor Cell Inoculations and Tumor Growth Measurement

Cells were harvested by trypsinization and washed in PBS. Mice were under isoflurane anesthesia during tumor injections. 5×10⁵ LLC cells were injected subcutaneously (s.c.) in 100 mL endotoxin-free PBS on the flank of recipient mice. 10⁵ 4T1 cells were injected orthotopically in the mammary fat pad of the mice. Tumor growth was measured using a digital caliper. Tumor volumes were measured biweekly and estimated by using the formula: Tumor volume=length×(width)² divided by 2, where length represented the largest tumor diameter and width represented the perpendicular tumor diameter. Intratumoral injections were performed using a 28G insulin syringe, when tumors had reached 100-150 mm³, using surgical forceps to hold the tumor constantly. For intravenous (i.v.) inoculations, a retro-orbital approach was adopted. Mice were anesthetized using inhaled isoflurane in a chamber. The eyeball was partially protruded from the socket by applying downward pressure to the skin dorsal and ventral to the eye. Injections were performed by placing the needle, bevel face-down, in order to decrease the likelihood of damaging the eyeball. Once the injection was complete, the needle was slowly and smoothly withdrawn. Triple antibiotic ophthalmic ointment was then applied to the eye.

Intraperitoneal injection was performed using a 28.5G insulin-syringe with the head tilted down. The needle was inserted at a 30° angle in the lower left or right quadrant. Transplantation of myeloma VQ4935 cells was performed via intracardiac injection after the 6-8 week old C57BL/6J recipient mice were sub-lethally irradiated at 6.0 Gy using an X-RAD 320 Irradiator. Intracardiac injection was performed by placement of needle in the fourth intercostal space and into the left ventricle. The needle was inserted at a 900 angle in the middle of the imaginary line connecting the sternal notch and xyphoid process serving as anatomical landmarks, and the needle was inserted slightly left of the sternum.

### Anti-PD1 Treatments

Recipient syngeneic mice (10 per arm) were injected with LLC-EV or LLC-Vkine cells (5×10⁵ cells per inoculum). Antibody treatments were with 100 μg of antibody in 100 μL of volume each (aPD1: Bio X Cell InVivoPlus, rat IgG2a, clone RPM1-14, Cat #BP0146, Lot #806321J2B; Isotype control: Bio X Cell InVivoPlus, rat IgG2a, clone 2A3, Cat #BE0089, Lot #796721M2). Treatments were administered on days 7, 10, and 14 post-inoculation. Tumor burden was tracked by measuring tumors with an electronic caliper every two days, beginning with Day 5 post-inoculation. Mice reached endpoint when they were found dead, were in clear distress, or when tumors reached 20 mm in any dimension. Animals found dead were considered to have reached endpoint on the off-day of measurements.

### Processing of Tumor Tissue

Unless stated otherwise, tumors were excised 21 days after transplantation. For subsequent analysis by flow cytometry, tumors were cut into pieces and digested with either Collagenase Ia (1 mg/mL) C2674 Sigma Aldrich and Hyaluronidase V (0.1 mg/mL) H6254 Sigma Aldrich for 40 min at 37 °C or with a mouse tumor dissociation kit (Miltenyi Biotec #130-096-730) using gentle MACS dissociator. Tissue was passed through a 70 mm cell strainer (Falcon) and washed with FACS buffer (PBS with 1% FCS) before proceeding with antibody staining. For RNA isolation, homogenization was performed in RLT buffer (QIAGEN) facilitated by a closed tissue grinder system (Fisher brand #02-542-09, 15 mL).

### Mass Cytometry

Tumor tissue was harvested and processed for mass cytometry analyses using the protocol described above for flow cytometry. After single cell suspensions were acquired, cells were washed with PBS, centrifuged at 300-400 g for 5 minutes and supernatant was discarded by aspiration. Cells were resuspended in PBS and Cell-ID Cisplatin (Fluidigm, #201064) was added to a concentration of 5 uM. After rigorous mixing, cells were incubated at room temperature for 5 minutes. Cells were then quench stained with MaxPar Cell Staining Buffer (Fluidigm, #201068) using 5× the volume of the cell suspension, centrifuged and supernatant was discarded by aspiration. The process was continued with surface staining. 50 ul of the antibody cocktail was added to each tube so the total staining volume was 100 ul (50 ul of cell suspension+50 ul antibody cocktail). Cells were stained for an hour at room temperature. All antibodies used for staining were either bought pre-conjugated to metal isotopes or were conjugated using the Maxpar Antibody Labelling Kit (Fluidigm, 201160B). Following incubation, cells were washed by adding 2 mL Maxpar Cell Staining Buffer to each tube, then centrifuged at 300×g for 5 minutes and supernatant was removed by aspiration. This step was repeated for a total of 2 washes, and cells were resuspended in residual volume by gently vortexing after final wash/aspiration. Cells were then fixed with 1.6% FA solution and incubated at room temperature for 10 minutes. Finally, cells were labelled with Cell-ID Intercalator-Ir (Fluidigm, #201192A) at a final concentration of 125 nM, incubated for an hour at room temperature and then analyzed on a Helios instrument (WB injector). All samples were resuspended in sufficient volume of 0.1 EQ beads (Fluidigm, #201078 by diluting one part beads to 9 parts Maxpar Cell Acquisition (CAS) solution.

Analysis of Mass Cytometry Data using viSNE

To visualize the immune contexture, the immune milieu of the tumor (CD45+) was enriched by manual gating among single events, equally subsampled to 6,000 events, then run through a Barnes Hut implementation of the t-SNE algorithm, viSNE, in the R package ‘Rtsne’, using optimized parameters (iterations:1000, perplexity:30, learning rate:455). To characterize the myeloid and lymphoid linages markers were selected for viSNE, excluding CD45.

### Flow Cytometry and Fluorescence-Activated Cell Sorting

Flow cytometric analyses were performed using an LSR II and/or LSR Fortessa X20. Data were analyzed using FlowJo (Tree Star). DAPI (0.5 mg/mL, Sigma-Aldrich) or a Live/Dead fixable cell stain (Ghost 780 Tonbo Biosciences) was used to exclude dead cells in all experiments, and anti-CD16/CD32 antibody (2.4G2) was used to block non-specific binding of antibodies via Fc-receptors. Quantification of total cell numbers by flow cytometry was done using fluorescent beads

(Biolegend Precision beads). For intracellular staining of IFNg and IL-2 in vitro, cells were treated with Golgi Plug (Brefeldin A 500×) and were collected 4 h later. Intracellular staining was performed in permeabilization buffer (eBioscience) for 30 min and cells were subsequently analyzed by flow cytometry. Sorting of tumor cells after retroviral transduction was done using a BD FACSAria or a BD FACSAria Fusion. Purity of cell populations was determined by re-analysis of a fraction of sorted cell samples.

Generation of iCD103 In Vitro

1.5×10⁶ BM cells were cultured in 10 mL RPMI1640 medium supplemented with 10% heat-inactivated FCS (Biochrom), penicillin/streptomycin and 50 mM b-mercaptoethanol. Recombinant human FLT3L (300-19, Peprotech) and recombinant murine GM-CSF (315-03, Peprotech) were added at day 0 of the culture. 5 mL complete medium was added between day 5 and day 6 to minimize apoptosis. Non-adherent cells were harvested on day 9, counted and re-plated at 3×10⁶ cells in 10 mL complete medium supplemented with FLT3L and GMCSF as on day 0. Non-adherent iCD103 were harvested on days 15-16. Cells were then validated by assaying for CD103, CD24, Clec9A, and CD11c by flow cytometry.

### ELISA

MutuDC1940 cells were left unstimulated or were in vitro stimulated with LPS for 8 or 24 hours at 37°. Cell-free supernatant was assessed for CXCL9 (R&D Quantikine mouse CXCL9 #MCX900) and IL27p28 (R&D Quantikine mouse IL27p28 #M2728) protein levels by ELISA according to the manufacturer's instructions (R&D). For the antigen-presentation assay, cell-free supernatants were collected and assessed for IFNg levels (R&D Quantikine mouse IFNg #P233156).

### Immunoblotting

Whole-cell lysates were prepared by boiling cells in Laemmli Sample Buffer (Bio-Rad) supplemented with 100 mM DTT for 10 min at a final concentration of 10⁷ cells per milliliter. A total of 10⁵ cells or 20 mg protein was resolved by SDS-PAGE and transferred to Immobilon-P PVDF membranes (Millipore). Membranes were blocked in 5% milk in TBS-T (25 mM Tris-HCl [pH 74], 0.13 M NaCl, 2.7 mM KCl). Primary antibodies (anti-HA [C29F4; Cell Signaling Technologies], anti-DPEAAE [PA1-1748A; Thermo]) were diluted in 5% milk-TBS-T, and membranes were incubated overnight at 4 °C. Secondary Ab-HRP conjugate, as well as anti-GAPDH-HRP conjugate (A00192; GenScript), incubations were carried out for 1 h at room temperature. Signal detection was achieved using Amersham ECL.

### Immunohistochemistry

Paraffin-embedded murine tumor sections and unstained 4-5 mm-thick human lung carcinoma TMA (US Biomax Inc., BC041115e) sections were deparaffinized and rehydrated using standard methods. Antigen retrieval was carried out in citrate buffer, pH 6.0 (Vector Laboratories, #H-3300) for DPEAAE and HA; and pH 8.0 for XCR1 and CD8 (Abcam, ab93680). Primary antibodies included αDPEAAE (PA1-1748A, Thermo Fisher), anti-HA (C29F4, Cell Signaling Technology), anti-XCR1 (D2F8T, Cell Signaling Technology), anti-CLEC9A (ab223188, Abcam), anti-NCR1 (NKp46) (MAB1850, R&D) and anti-CD8 (C8/144B, Ebioscience). The αDPEAAE neoepitope

antibody has been previously validated. Stained slides were examined using an Echo Revolve microscope with attached digital camera. αDPEAAE immunostaining score was assessed by scoring staining intensity (0 for no staining, 1 for low/weak staining, 2 for moderate staining and 3 for strong/intense staining).

Dual staining XCR1/CLEC9A on human tonsil was performed by the UW TRIP lab as follows: The experiment was run on Roche Ventana Medical System's Discovery Ultra Automated platform. Deparaffinization was carried out on the instrument, as was heat induced epitope retrieval in the form of “cell conditioning” with CC1 buffer (Ventana #950-500), a Tris based buffer pH 8.4 for approximately 56 minutes at 95 °C. Slide was incubated with the first primary antibody XCR1 diluted 1:40 in DaVinci Green antibody diluent (BioCare Medical #PD900H) for 60 min at 37°. Slide was rinsed with Reaction Buffer (Ventana #950-300) for the preset time and duration. Discovery OmniMap anti-Rabbit HRP (Ventana #760-4311) was applied for 16 min at 37°. Slide was rinsed with Reaction Buffer (Ventana #950-300) for the preset time and duration. Discovery ChromoMap DAB detection (Ventana #760-159) was applied for the preset time. Denaturing agent in the form of Discovery Inhibitor (Ventana #760-4840) was applied for the preset time. Slide was incubated with the second primary antibody CLEC9A diluted 1:50 in Ventana antibody diluent with casein (Ventana #760-219) for 60 min at 37°. Slide was rinsed with Reaction Buffer (Ventana #950-300) for the preset time and duration. Discovery OmniMap anti-Rabbit HRP (Ventana #760-4311) was applied for 16 min at 37°. Slide was rinsed with Reaction Buffer (Ventana #950-300) for the preset time and duration. Discovery Teal HRP detection kit (Ventana #760-247) was applied for 32 minutes. Slide was removed from the instrument and rinsed with dawn dish soap and warm tap water followed by rinsing with dH2O. Slide was counterstained with Harris hematoxylin (1:5 diluted in dH2O) for 45 seconds. Slide was rinsed with dH2O. Slide was dehydrated in the oven (60 degrees C) followed by dipping in Xylene. Slide was cover slipped.

Triple staining DPEAAE/XCR1/CD8+ on lung cancer TMA was performed by the UW TRIP lab as follows: The experiment was run on Roche Ventana Medical System's Discovery Ultra Automated platform. Deparaffinization was carried out on the instrument, as was heat-induced epitope retrieval in the form of “cell conditioning” with CC1 buffer (Ventana #950-500), a Tris based buffer pH 8.4 for approximately 56 minutes at 95 °C. Slide was incubated with the first primary antibody XCR1 diluted 1:25 in DaVinci Green antibody diluent (BioCare Medical #PD900H) for 60 min at 370, Slide was rinsed with Reaction Buffer (Ventana #950-300) for the preset time and duration. Discovery OmniMap anti-Rabbit HRP (Ventana #760-4311) was applied for 16 min at 37 degrees. Slide was rinsed with Reaction Buffer (Ventana #950-300) for the preset time and duration. Discovery ChromoMap DAB detection (Ventana #760-159) was applied for the preset time. Denaturing agent in the form of Discovery Inhibitor (Ventana #760-4840) was applied for the preset time. Slide was incubated with the second primary antibody DPEAAE diluted 1:800 in Ventana antibody diluent with casein (Ventana #760-219) for 28 min at 37 degrees. Slide was rinsed with Reaction Buffer (Ventana #950-300) for the preset time and duration. Discovery OmniMap anti-Rabbit HRP (Ventana #760-4311) was applied for 16 min at 37 degrees. Slide was rinsed with Reaction Buffer (Ventana #950-300) for the preset time and duration. Discovery Teal HRP detection kit (Ventana #760-247) was applied for 32 minutes. Denaturing agent in the form of Discovery Inhibitor (Ventana #760-4840) was applied for the preset time. Slide was incubated with the third primary antibody CD8 (pre-diluted ready to use) for 16 min at 37 degrees. Slide was rinsed with Reaction Buffer (Ventana #950-300) for the preset time and duration. Discovery OmniMap anti-Rabbit HRP (Ventana #760-4311) was applied for 16 min at 37 degrees. Slide was rinsed with Reaction Buffer (Ventana #950-300) for the preset time and duration. Discovery Purple HRP detection kit (Ventana #760-229) was applied for 16 minutes. Slide was removed from the instrument and rinsed with dawn dish soap and warm tap water followed by rinsing with dH2O. Slide was counterstained with Harris hematoxylin (1:5 diluted in dH2O) for 45 seconds. Slide was rinsed with dH2O. Slide was dehydrated in the oven (60 degrees C) followed by dipping in Xylene. Slide was cover slipped. An Olympus BX43 microscope with 40× objective (400× magnification) was used for data points reported as per “high power field”.

### Imaging and Morphometric Analysis of XCR1+CLEC9A Colocalization

Imaging of a human tonsil slide was performed on a Vectra 2 multispectral scanner (Akoya Biosciences). The stained slide was then loaded onto the instrument and 24 8-bit Bright Field 20× images were acquired for analysis. A customized spectral library algorithm for all chromogens and counterstain was created using Nuance Software version 3.0.2 (PerkinElmer). The inForm software version 2.4.7 was used to segment tissue subcellular compartments (nucleus, cytoplasm or membrane), and to measure biomarker expression. The double positivity algorithm was used to measure colocalization of cells expressing XCR1 and CLEC9A as double-positive percentage rate.

### RNA Isolation and Quantitative Real-Time PCR

RNA was isolated using QIAGEN RNeasy Mini Kit and cDNA was synthesized using the iScript Reverse Transcription Supermix (Biorad). Quantitative real-time (qRT-PCR) analysis was performed using SsoAdvanced Universal SYBR Green Supermix (Biorad #1725272) according to the manufacturer's instructions on an CFX96 Touch Real Time PCR detection (Biorad) using the relative standard curve method. PCR conditions were 2 min at 50 °C, 10 min at 95 °C followed by 40 2-step cycles of 15 s at 95 °C and 1 min at 60 °C. Primers for the targets as well as SDHA for normalization control, were used to assess relative gene expression. For DMXAA-response analysis, RT2 Profiler PCR Array (QIAGEN, Cat. no. PAMM-021Z) was used. In brief, RNA was isolated from tumors and was reverse transcribed using kits mentioned above. cDNA was mixed with RT² SYBR® Green qPCR Mastermix (Cat. no. 330529). The mixture was aliquoted across the RT2 Profiler PCR Array (in 96-well format) and was run on the Real Time PCR machine. Data analysis was performed using the manufacturer's online platform for RT2 Profiler Data analysis software.

### Generation of Versikine Cell Culture Supernatant

5×10⁶ HEK293 and HEK-Vkine expressing cells were seeded in T-175 cell culture flasks and cultured in DMEM 10% FBS media. After 75 to 80% confluency, the cell media was changed to DMEM 1% FBS media. Subsequently, media supernatant was collected after 48 hours of incubation. The collected supernatant was centrifuged to remove debris and filtered with 0.45 m filter. The filtered supernatant was then concentrated 30 times to the initial volume using Sartorius Vivaspin 20, 10,000MWCOPES concentrator (Cat. No. VS2001). Endotoxin assay was performed using Genscript ToxinSensor Gel Clot Endotoxin Assay Kit (Cat. No. L00351) according to the manufacturer's instructions to rule out contamination. The presence of versikine in concentrated supernatant was confirmed using western blot using c-Myc Antibody (Novus Bioc-Myc Antibody (9E10)—Chimeric NBP2-52636). Concentrated supernatant containing versikine was then used to treat MutuDC1940 cells. 2×10⁵ MutuDC1940 cells per well were seeded in 12 well plate. The following day, 10% supernatant was added to the plate media. Cells were then incubated for 72 hours. After incubation, total RNA was extracted from MutuDC1940 cells.

### Library Preparation for RNA-seq

A total amount of 1 mg RNA per sample was used as input material for the RNA sample preparations. Sequencing libraries were generated using NEBNext® Ultra™ RNA Library Prep Kit for Illumina® (NEB, USA) following manufacturer's recommendations and index codes were added to attribute sequences to each sample. Briefly, mRNA was purified from total RNA using poly-T oligo-attached magnetic beads. Fragmentation was carried out using divalent cations under elevated temperature in NEBNext First Strand Synthesis Reaction Buffer (5×). First strand cDNA was synthesized using random hexamer primer and M-MuLV Reverse Transcriptase (RNase H-). Second strand cDNA synthesis was subsequently performed using DNA polymerase I and RNase H. Remaining overhangs were converted into blunt ends via exonuclease/polymerase activities. After adenylation of 30 ends of DNA fragments, NEBNext Adaptor with hairpin loop structure were ligated to prepare for hybridization. In order to select cDNA fragments of preferentially 150-200 bp in length, the library fragments were purified with AMPure XP system (Beckman Coulter, Beverly, USA). Then 3 mL USER Enzyme (NEB, USA) was used with size-selected, adaptor-ligated cDNA at 37 °C for 15 min followed by 5 min at 95 °C before PCR. Then PCR was performed with Phusion High-Fidelity DNA polymerase, Universal PCR primers and Index (X) Primer. At last, PCR products were purified (AMPure XP system) and library quality was assessed on the Agilent Bioanalyzer 2100 system. The clustering of the index-coded samples was performed on a cBot Cluster Generation System using PE Cluster Kit cBot-HS (Illumina) according to the manufacturer's instructions. After cluster generation, the library preparations were sequenced on an Illumina platform and paired-end reads were generated.

### RNA-Seg Data Analysis

Raw data (raw reads) of FASTQ format were firstly processed through fastp. In this step, clean data (clean reads) were obtained by removing reads containing adapter and poly-N sequences and reads with low quality from raw data. At the same time, Q20, Q30 and GC content of the clean data were calculated. All the downstream analyses were based on the clean data with high quality. Reference genome and gene model annotation files (GRCm38) were downloaded from genome website browser (NCBI/UCSC/Ensembl) directly. Paired-end clean reads were aligned to the reference genome using the Spliced Transcripts Alignment to a Reference (STAR) software (v2.6.1d). FeatureCounts (v1.5.0-p3) was used to count the read numbers mapped of each gene. RPKM of each gene was calculated based on the length of the gene and reads count mapped to this gene. Differential expression analysis between two conditions/groups (three biological replicates per condition) was performed using DESeq2 R package (v1.20.0). DESeq2 provides statistical routines for determining differential expression in digital gene expression data using a model based on the negative binomial distribution. The resulting p values were adjusted using the Benjamini and Hochberg's approach for controlling the False Discovery Rate (FDR). Genes with an adjusted p value <0.05 found by DESeq2 were assigned as differentially expressed.

### Gene Set Enrichment Analysis

Gene Set Enrichment Analysis was performed by comparing MutuDC1940-Vkine (treated with PBS, 4 h) RNA-seq data to the corresponding MutuDC1940-EV sample. 4736 differentially expressed gene features for each condition were ranked by the signal to noise metric of GSEA and the analysis was performed using the standard weighted enrichment statistic against human gene sets contained in the Molecular Signatures Database (MSigDB v7.4) that included all (H) Hallmark gene sets, (C2) curated gene sets, and (C3) motif gene sets. The normalized enrichment score (NES) was calculated using 1000 gene set permutations.

### NK Cell Depletion In Vivo

For depletion of NK cells, mice were injected i.p. with 50 μg of anti-asialoGM1 (Wako Pure Chemical Industries, 100 mL/mouse) on days −1, 0, 7, 14 around tumor inoculation.

### MutuDC1940 Apoptosis Assay

2×10⁶ MutuDC1940-EV or -Vkine cells were plated per well in a 6-well plate and allowed to attach overnight at 37 °C in 5% CO2 incubator. Next day, cells were pretreated with 10 ng/mL murine GM-CSF (Peprotech, catalog no #315-03) for 6 h, followed by staurosporine [(AM-2282), Selleckchem, Catalog no. S1421] treatment at indicated doses for 24 h. Next day, for apoptosis assay, cells and medium were harvested, washed twice with cold PBS and then resuspended in Annexin-V binding buffer. The cell suspension was stained with APC Annexin V and 7-AAD viability dye according to manufacturer's instructions (APC Annexin V Apoptosis Detection Kit with 7-AAD, Biolegend, catalog #640930). Each sample was analyzed by flow cytometry with proper machine settings.

### Antigen Presentation Assay

MutuDC1940 were cultured and treated with LPS or PBS control respectively overnight. Next day, the cells were harvested and plated on 96-well round bottom plates at a density of 100,000 cells per plate. DC were then loaded with OVA peptide 257-264 (SIINFEKL) (3 ng/mL) and incubated for 4 hours at 37 °C. MutuDC1940 were then washed with 0.1% PBS-BSA and centrifuged at 800×g and were fixed with 50 mL per well of freshly made PBS-glutaraldehyde (GTA) 0.008% (vol/vol) and incubated for 5 minutes on ice. 50 mL of PBS-glycine 0.4M was added to the PBS-GTA 0.008% solution and cells were centrifuged at 800×g for 2 minutes at 4 °C. Plates were subsequently flicked. Finally, 100 mL of PBS-glycine 0.2M was added to each well and centrifugation of the plates at 800×g for 2 min at 4 °C followed. Fixed DC were then washed twice with 200 mL/well of T cell culture medium (RPMI 1640 containing 10% heat inactivated FBS, 100 IU/mL penicillin, 100 mg/mL streptomycin, 2 mM glutamax, 50 mM β-mercaptoethanol, 1×MEM nonessential amino acids, 1× sodium pyruvate) before being resuspended in 100 ul/well of the same medium. 100,000 OT-I T cells per well in 100 mL T cell culture medium were added (to a final volume of 200 mL). The co-cultured OT-I T cells with the cross-fixed DCs were incubated for 18 h at 37 °C. Cell activation cocktail with brefeldin A (PMA/Ionomycin and Brefeldin A Biolegend, #423303) was added to the wells 4 hours before harvesting. At the time of the harvest, plates were spun down at 800×g for 2 minutes at 4 °C and supernatant was kept for subsequent cytokine analysis. They were then washed with 100 mL of 0.1% PBS-BSA before proceeding with live/dead staining with fixable viability Ghost 780 dye (Tonbo Biosciences) for 30 min at 4 °C in PBS. Cells were then washed with 0.1% PBS-BSA and stained with a cocktail of 70 mL/well of the surface markers (CD8a and CD3) for 40 minutes at 4 °C. They were then fixed and permeabilized using the eBiosciences fixation and permeabilization buffer set (eBioscience 88-8824-00) according to the manufacturer's instructions followed by intracellular staining of IFNγ and IL-2 in permeabilization buffer. Finally, cells were washed 2 times with 0.1% PBS-BSA, centrifuged and resuspended in 100 mL/well of PBS-BSA and then analyzed by flow cytometry.

cDC1 and VCAN Landscape Analysis of TCGA Datasets

Level 4 gene expression data were downloaded from the TCGA Data Portal and filtered to retain only cancer types of known epithelial origin for a total of 7591 samples across 20 different cancer types. Single-sample Gene Set Enrichment Analysis (ssGSEA) was performed to measure the signature of gene sets designed to measure overall immune infiltration, cDC1 density and CD8+ T cell density. TCGA samples were grouped by cancer type and sorted based on median expression of versican (VCAN) and median cDC1 signature. To measure cancer-specific relationships between VCAN expression and the cDC1 signature an ordinary least squares linear model was fit on these two variables to measure their relationship within each cancer type. Nominal p values from these 20 different models were corrected for multiple hypothesis testing using the Benjamini-Hochberg method and q-values less than 0.1 were considered statistically significant.

### Computational Modeling of CD8+ T Cell Density and Versikine Response Signature

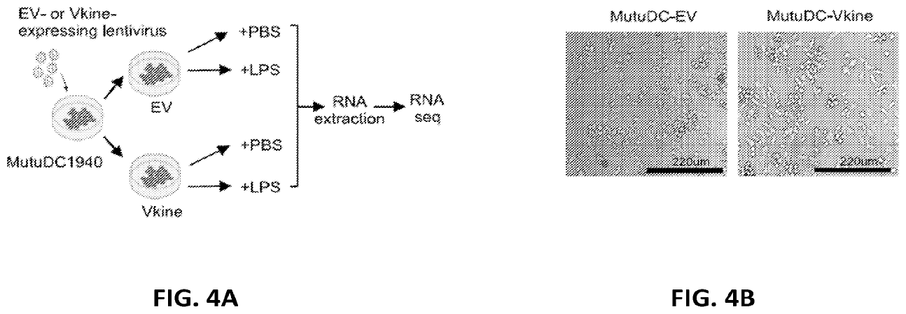

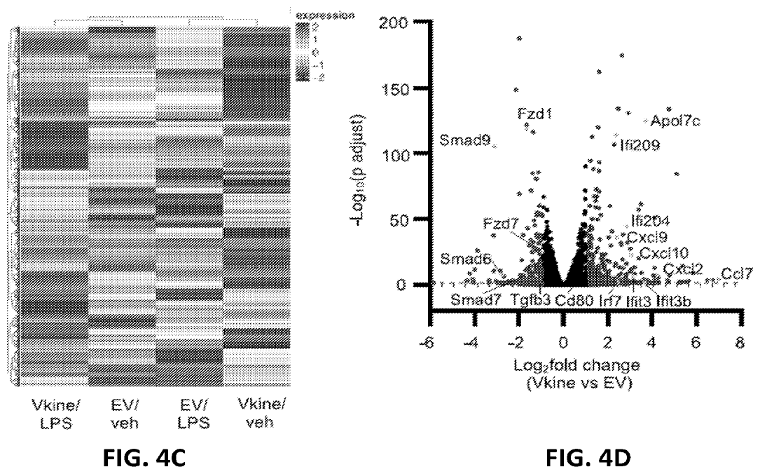

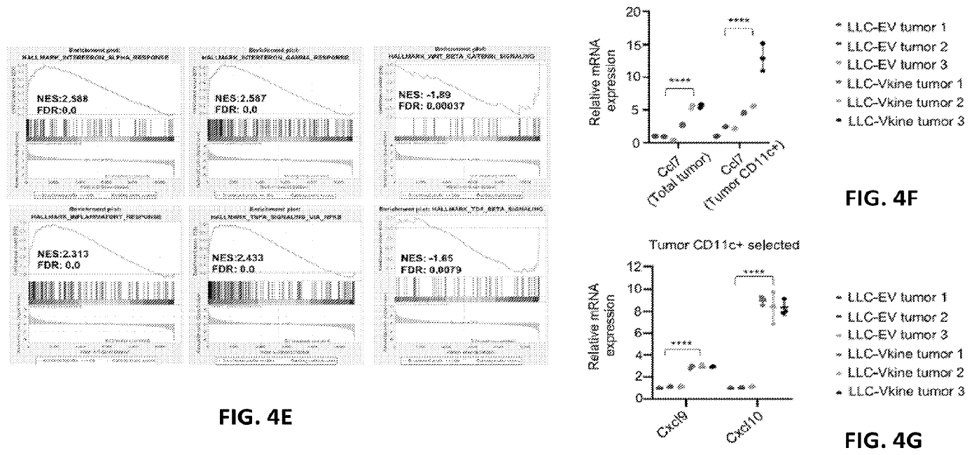

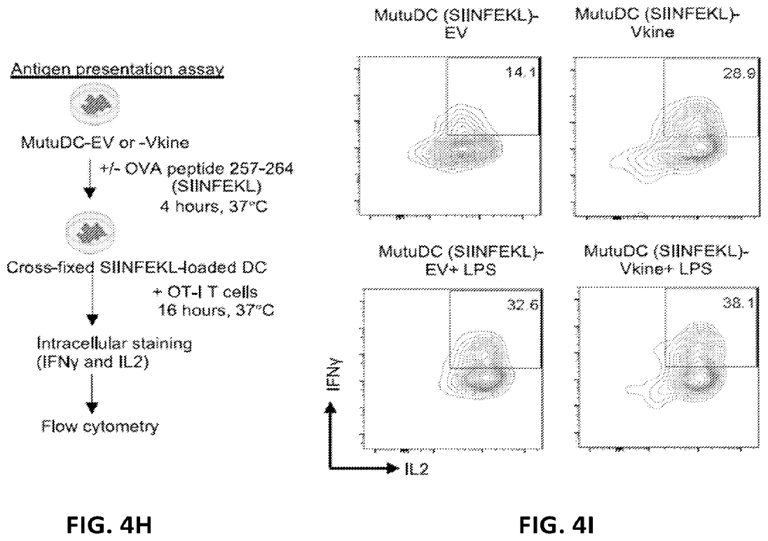

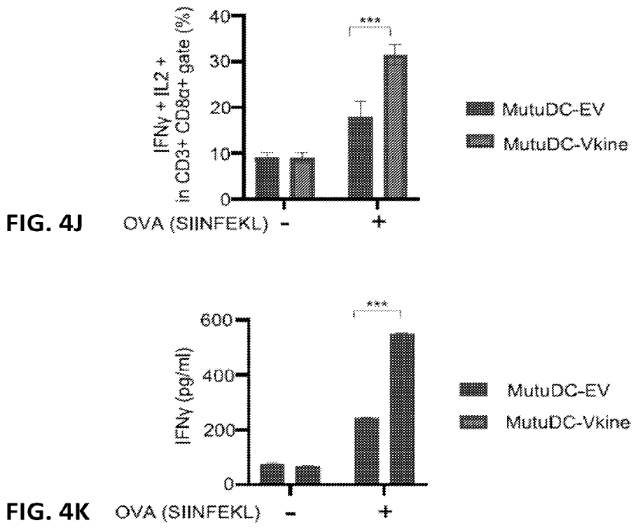

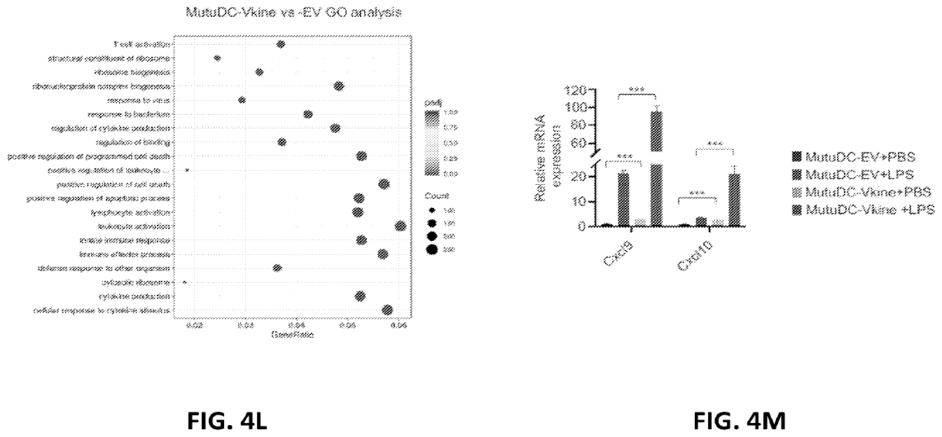

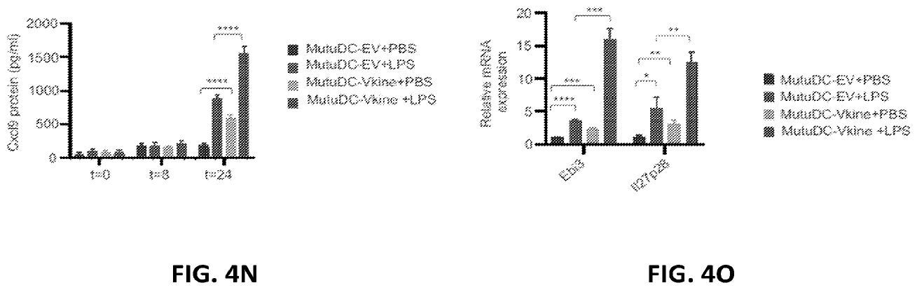

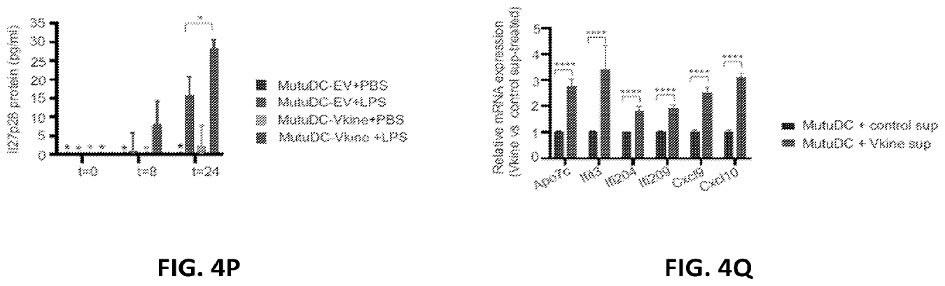

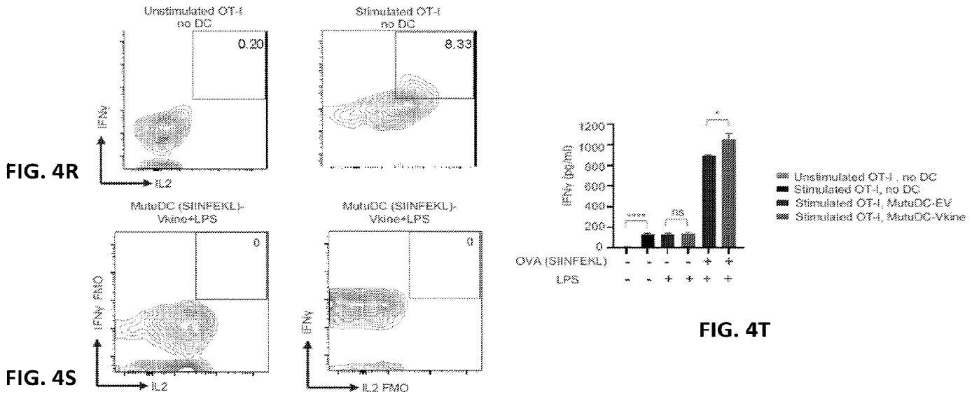

Differential expression using DESeq2 was performed to identify genes that were differentially expressed between the PBS-EV and PBS-versikine conditions (FIGS. 4A and 4C). Genes with a q-value of less than 0.1 were considered significant. Two gene sets were defined to measure the response to versikine by selecting the 100 genes more significantly induced (versikine-up) and most significantly repressed (versikine-down). These mouse genes were then mapped to their human orthologues using the HGNC Comparison of Orthology Predictions (HCOP) tool. ssGSEA was then used to measure the signature of these two gene sets in the 1017 lung samples in the TCGA cohort and overall versikine response level was summarized as the difference between versikine-up and versikine-down signature levels. This versikine response signature was then compared to the CD8 effector T cell signature using an ordinary least squares linear model including the overall immune infiltration signature as a covariate. p-values of less than 0.05 were considered significant.

### Example 1—Conventional Type Dendritic Cells (cDC1s) Localize in Peritumoral Stroma and are Regulated by the VCAN Pathway

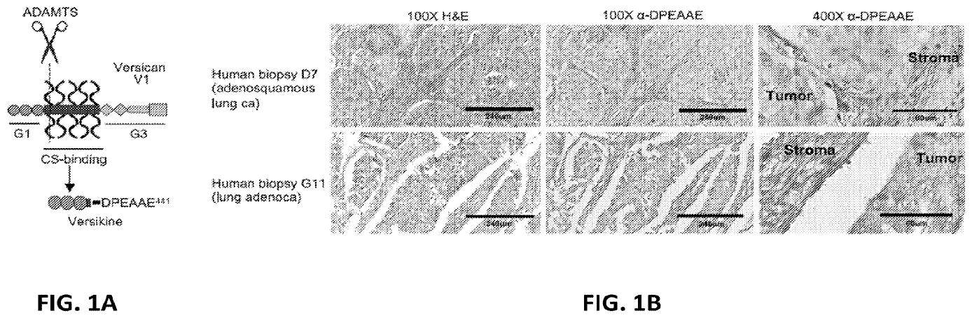

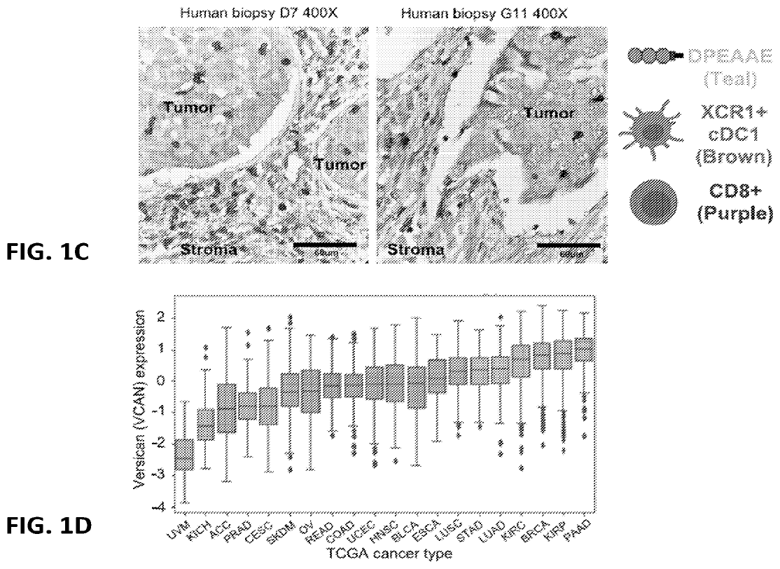

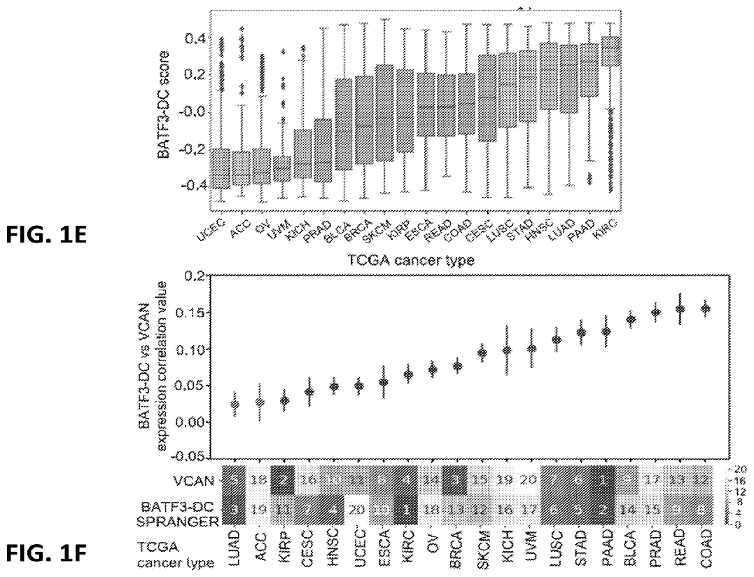

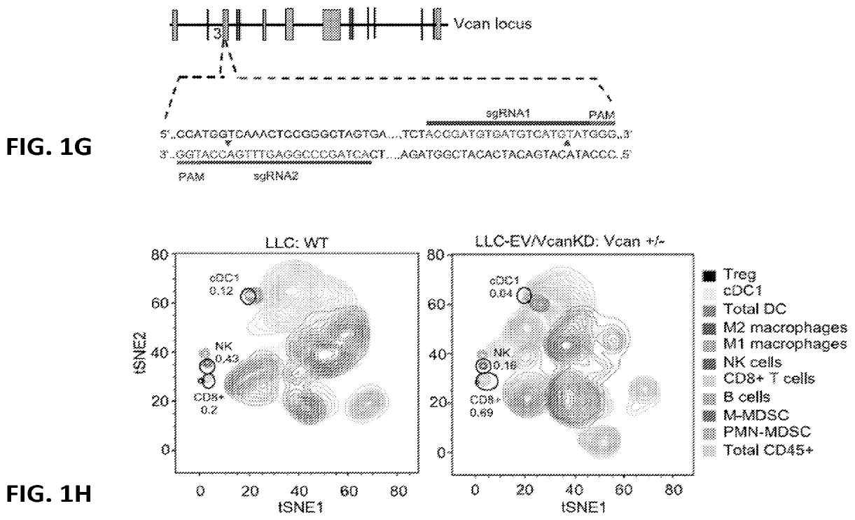

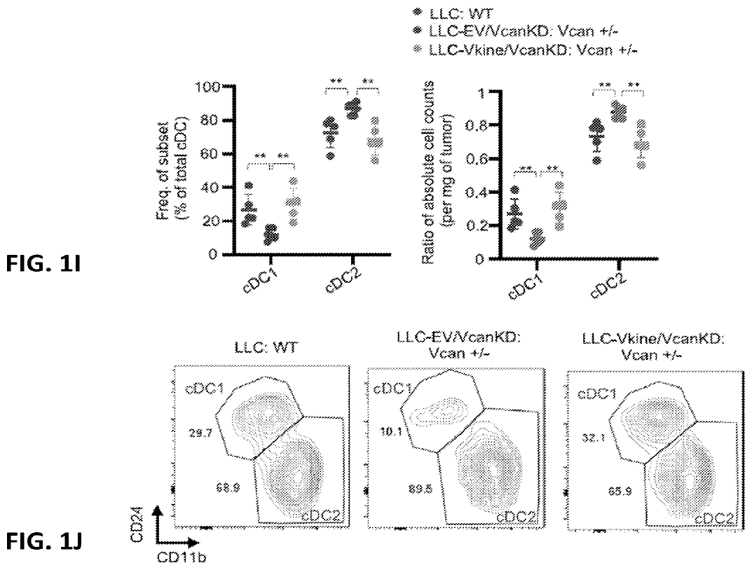

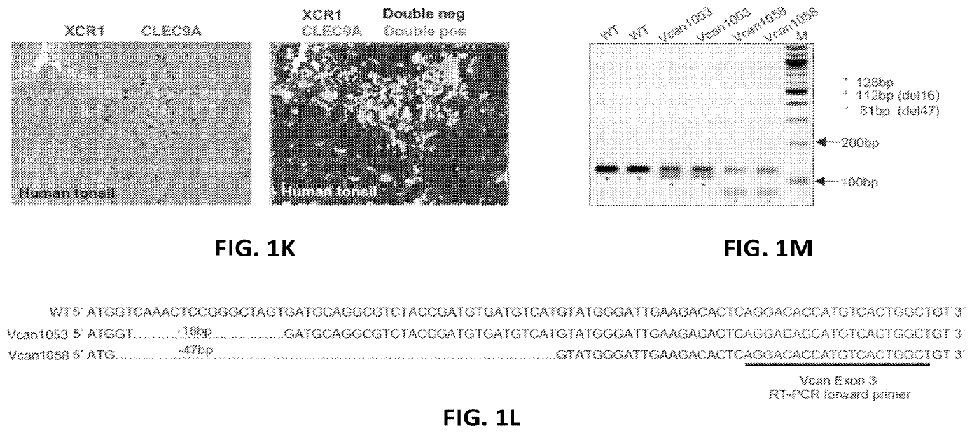

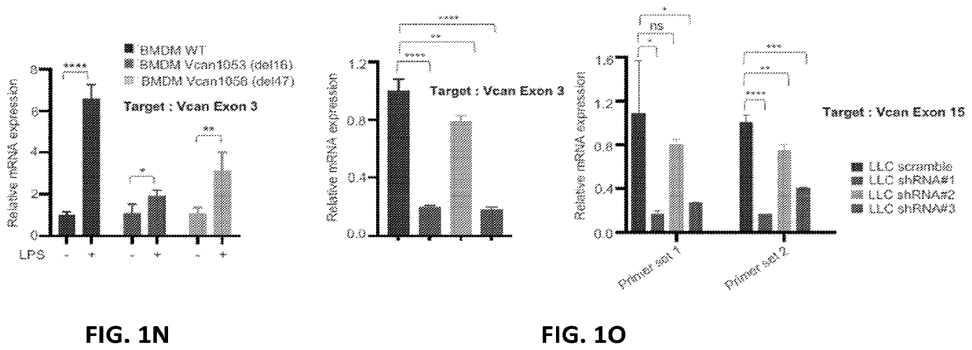

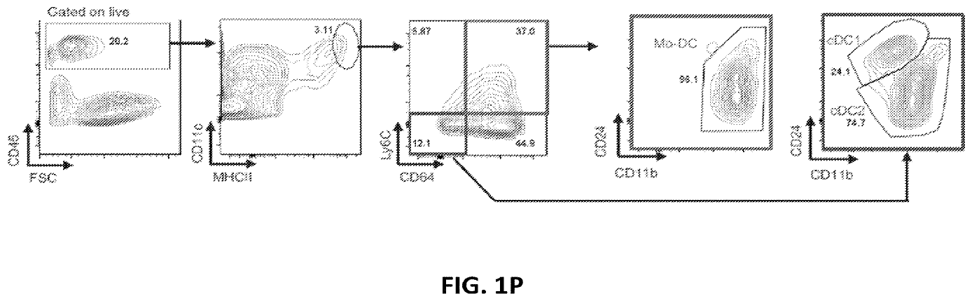

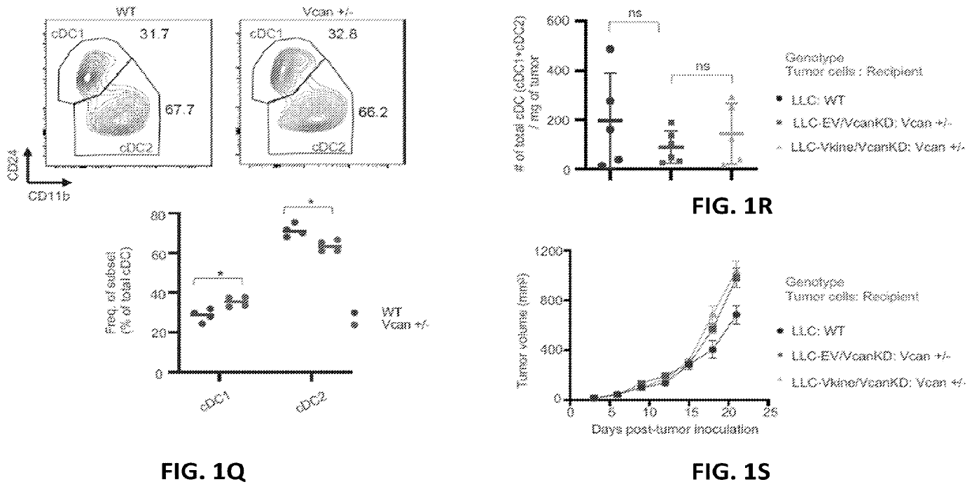

Versican (VCAN) and its binding partner hyaluronan are cardinal components of the provisional extracellular matrix in development, wound healing, and cancer. In tumors, stromal provisional matrix is thought to coordinate critical pro-tumor functions (e.g., angiogenesis) and prime conversion toward collagen-rich, desmoplastic stroma. For deposition into stromal matrix, VCAN is sourced from stromal mesenchymal cells, immune infiltrating cells (particularly myeloid cells, such as macrophages), and, in some cases (such as in lung carcinomas), the tumor cells themselves. However, VCAN proteolytic processing is located primarily in stroma because of the local activity of stromal fibroblast-derived ADAMTS (a disintegrin and metalloproteinase with thrombospondin motifs; a family of multidomain extracellular protease enzymes) VCANases (enzymes which cleave versican). Using an immunohistochemistry (IHC)-validated antibody against DPEAAE, a neoepitope generated through VCAN proteolysis at Glu441-Ala442 (V1 isoform) (FIG. 1A), VCAN proteolysis signal was observed in approximately 83% of human lung cancer cases in a stromal distribution (FIG. 1B). To determine the location of cDC1s relative to sites of stromal VCAN proteolysis, multiplex IHC was performed with antibodies detecting the cDC1 lineage marker XCRT and CD8. Remarkably, even in T cell-inflamed tumors demonstrating intra-epithelial CD8+ T cell penetration, XCR1+ cells were confined within stromal sheets recurrently undergoing VCAN proteolysis (FIG. 1C). The antibody against XCR1 has been previously validated, and close correlation between XCR1 staining and the signal for the cDC1 lineage marker CLEC9A was confirmed in human tonsils (FIG. 1K).

Matrikines (such as versikine) have been defined as “peptides liberated by partial proteolysis of extracellular matrix macromolecules which are able to regulate cell activities not triggered by their full-size parent macromolecules”. Notwithstanding its distinct neo-activity, versikine ultimately derives from parental VCAN through ADAMTS proteolysis (FIG. 1A); therefore, it was hypothesized that VCAN expression (the substrate for versikine) and cDC1 abundance correlate in human cancer. VCAN gene expression and cDC1 signature scores were compared across 7,591 samples from 20 The Cancer Genome Atlas (TCGA) cancer types (FIGS. 1D and 1E). A significantly positive correlation between VCAN expression and cDC1 signature scores was observed in several human carcinomas (FIG. 1F), suggesting that the VCAN pathway broadly regulates cDC1s.

To dissect the relevant mechanisms, novel Vcan-targeted models were generated that disrupt exons coding for Vcan's N terminus (Vcan exons 2-6). The widely used Vcan^{hdf} null mutant (hdf, heart defect) targeting exon 7 is embryonic lethal in homozygosity. Vcan^{hdf} hemizygosity demonstrates functional haploinsufficiency for CD8+-mediated control of viral infection. CRISPRCas9-mediated mutagenesis was used to disrupt exon 3 sequences, abolishing transcription of all Vcan isoforms (and, consequently, generation of versikine) (FIG. 1G). Two founders (Vcan1053 and Vcan1058) bearing Vcan exon 3 deletions were found (16 bp and 47 bp, respectively) (FIGS. 1L and 1M). Defective Vcan message induction was confirmed after stimulation of BM-derived macrophages (BMDM) with the Toll-like receptor (TLR)-4 agonist lipopolysaccharide (LPS) (FIG. 1N). BMDMs stimulated with LPS preferentially transcribe the V1 isoform, the precursor to versikine. The Vcan1053 transgenic line demonstrated the most severe defect in Vcan message induction (hereafter designated Vcan^{+/-}).

Lewis lung carcinoma (LLC) cells produce Vcan cell autonomously. Endogenous Vcan expression was knocked down in LLC cells using short hairpin RNA (shRNA) targeting Vcan exon 8 (encoding for the glycosaminoglycan (GAG)β domain in the VCAN-V1 isoform, the major isoform produced in LLC; FIG. 1A), hereafter referred to as LLC^{VcanKD}. The reduced transcription of Vcan was validated in LLCVcanKD cells using 5′ and −3′ Vcan primers, as shown in FIG. 1O. The intratumoral immune contexture was characterized in LLC^{VcanKD} tumors implanted in Vcan^{+/-} mice through mass cytometry and compared it with wild-type (WT) controls (FIG. 1H). Vcan depletion resulted in expansion of CD8+ T cells, consistent with the known role of non-proteolyzed VCAN in T cell exclusion. In keeping with the hypothesis, cDC1 loss was observed in Vcan-depleted tumors (FIG. 1H). To corroborate the mass cytometry findings, intratumoral DCs were delineated through 9-color flow cytometry (FIG. 1P). cDC1s were depleted in LLC^{VcanKD} tumors implanted into Vcan^{+/-} mice (FIGS. 1I and 1J). In contrast, steady-state splenic cDC1s were not reduced (in fact, they were mildly increased) in Vcan+/−mice (FIG. 1Q). Ectopic expression of versikine in LLCVcanKD cells restored near-physiological cDC1 abundance (FIGS. 1I and 1J). Intratumoral DC absolute count ratios corroborated the cell frequency findings (FIGS. 1I and 1J) despite fluctuations in total cDC−counts using the collagenase/hyaluronidase tumor dissociation protocol delineated in the STAR Methods (FIG. 1R). Vcan depletion and versikine add-back did not affect tumor growth rates (FIG. 1S). These results demonstrate that VCAN is necessary and that its proteolytic product versikine is sufficient for cDC1 abundance in the TME.

### Example 2—VCAN Matrikine Versikine Promotes cDC1 Abundance In Vivo

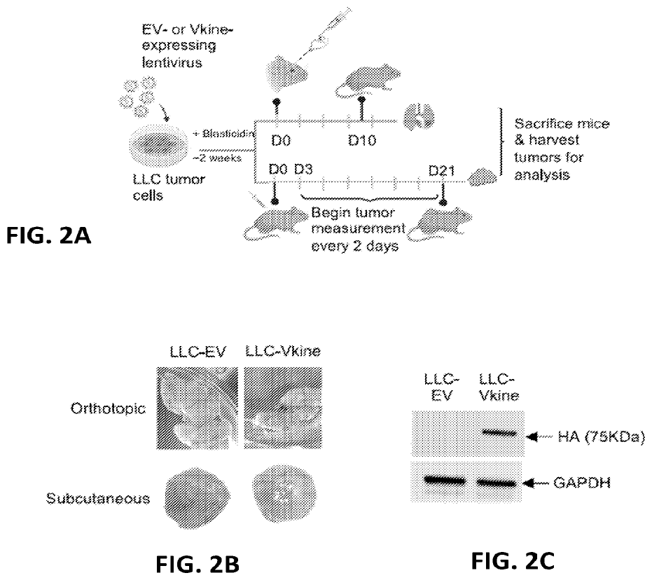

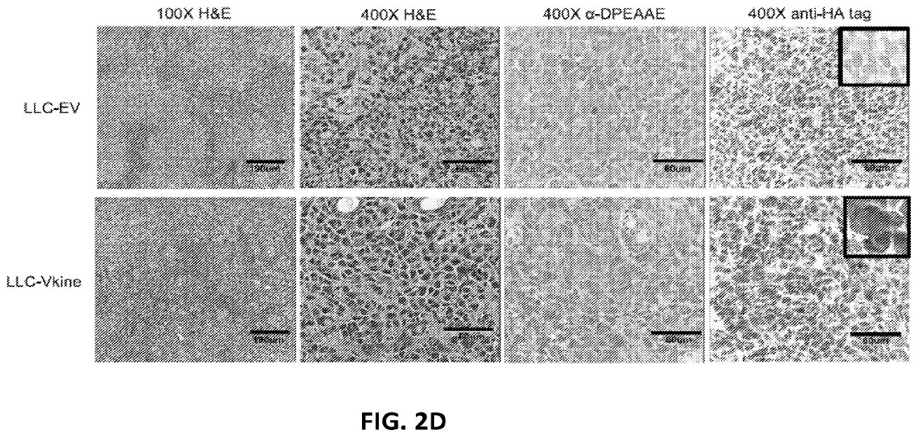

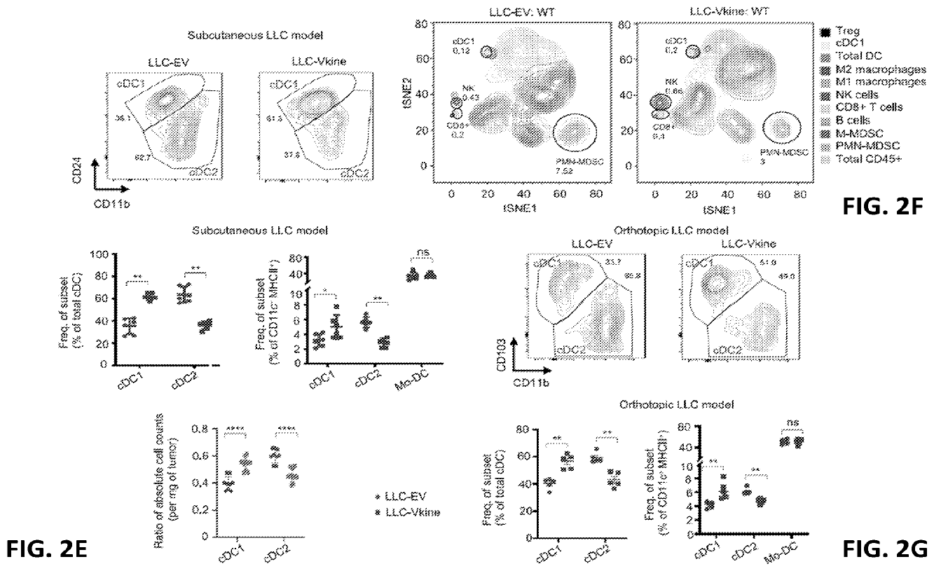

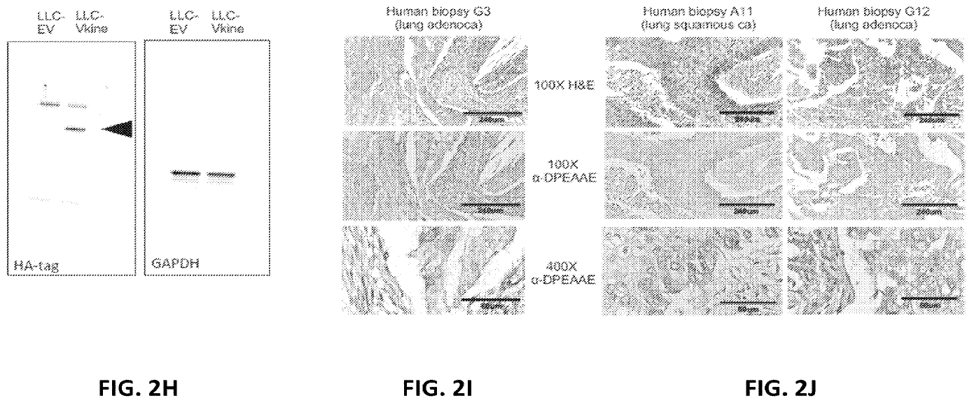

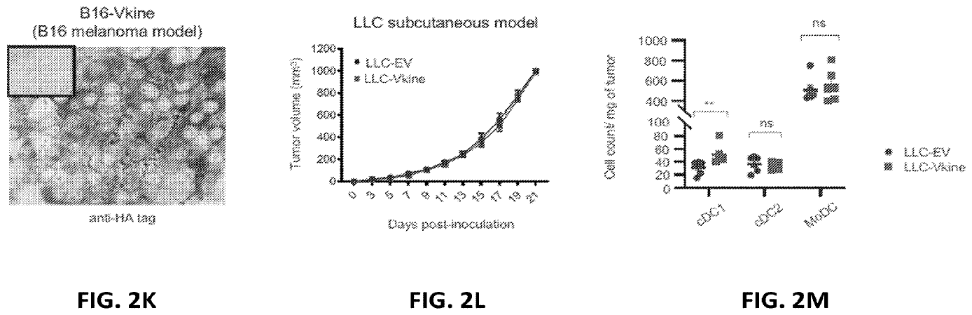

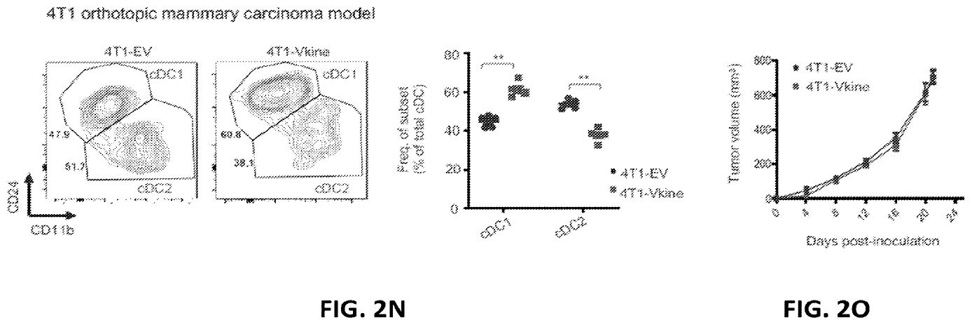

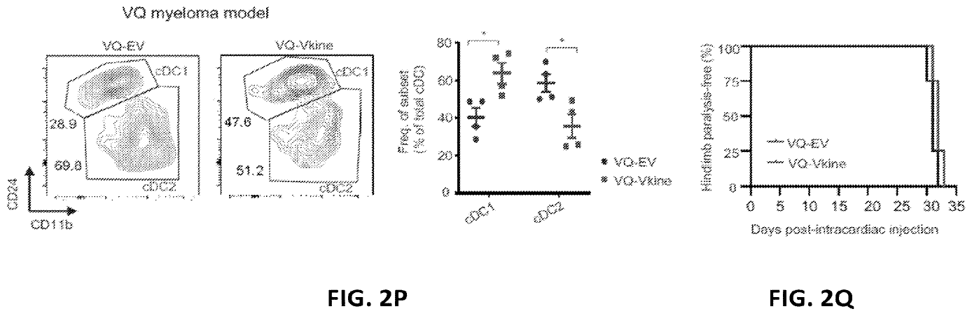

VCAN proteolysis is a composite event that produces two simultaneous, coupled consequences: first, parental VCAN clearance, and second, the novel activities of the released matrikine versikine. To uncouple versikine's activity from the effects of parental VCAN depletion, LLC cells stably expressing hemagglutinin (HA)-tagged versikine in the WT background were generated (FIG. 2A). Expression of versikine in LLC cells did not result in grossly visible increases in angiogenesis or hemorrhagic propensity (FIG. 2B). Ectopically expressed versikine was readily detectable by anti-HA tag western blotting in murine tumor lysates at approximately 75 kDa (FIGS. 2C and 2H).

Murine implantable tumor models do not recapitulate the human architecture of epithelial nests and stromal sheets; this limitation has been attributed to acquisition of mesenchymal features through successive passaging (FIG. 2D). However, the LLC model does retain physiological relevance because of its tumor-intrinsic production of Vcan that regulates myeloid cells in the TME. In this regard, LLC mimics a subset of human lung cancers with detectable VCAN production and processing in stromal and epithelial compartments (FIG. 2I-2J). Ectopically expressed versikine was detected in a membranous distribution consistent with its accumulation in the pericellular glycocalyx (FIG. 2D), a physiological site of VCAN cleavage. Membranous localization of ectopic versikine was also seen in B16 melanoma tumor cells that transcribe a very low to undetectable endogenous Vcan message (FIG. 2K).

There were no differences in the growth rates between LLC-empty vector (EV) and LLC-versikine (LLC-Vkine) tumors (FIG. 2L). cDC populations were analyzed by conventional flow cytometry (FIG. 1P). cDC1 expansion was confirmed in LLC-Vkine tumors (FIG. 2E), notably the opposite phenotype to that of Vcan depletion (FIG. 1J), as well as unchanged monocytic-derived dendritic cell (Mo-DC) frequency (FIG. 2E). In the later stages of the study, optimized tumor cell dissociation protocols permitted determination of absolute DC counts; only cDC1s expanded in absolute terms (FIG. 2M). By mass cytometry, in addition to cDC1 accumulation, expansion of an innate lymphoid NKT.1+NKp46+ population, an increase in intratumoral CD8+ T cells, as well as polymorphonuclear myeloid-derived suppressor cell (PMN-MDSC) depletion was observed (FIG. 2F), the latter being consistent with IRF8 regulation by versikine. To confirm cDC1 expansion in the orthotopic lung milieu, EV and versikine (Vkine)-expressing LLC cells were injected intravenously to seed the relevant lung microenvironment and lungs bearing metastatic deposits were harvested on day 10 (FIG. 2G). LLC-Vkine tumors within the relevant lung microenvironment also showed enhanced cDC1s (FIG. 2G). As with prior reports, CD103 was found to be more consistent compared with CD24 for cDC1 enumeration in native lung tissue.

Versikine promoted cDC1s in the BALB/c-derived, orthotopic 4T1 mammary carcinoma model (FIG. 2N). Growth rates of 4T1 versikine-replete tumors did not differ from their EV counterparts (FIG. 2O). Earlier a role of VCAN proteolysis was reported in shaping the human BM myeloma immune microenvironment. More recently, the first Ras-driven immunocompetent myeloma model was developed, VQ. Versikine-replete VQ myeloma tumors demonstrated enhanced cDC1s (FIG. 2P). Myeloma clinical progression was unaffected by versikine (FIG. 2Q). Therefore, versikine promotes cDC1 abundance in solid and hematopoietic cancers across murine genetic backgrounds.

### Example 3—Pre-DC Differentiation is Unaffected by Versikine

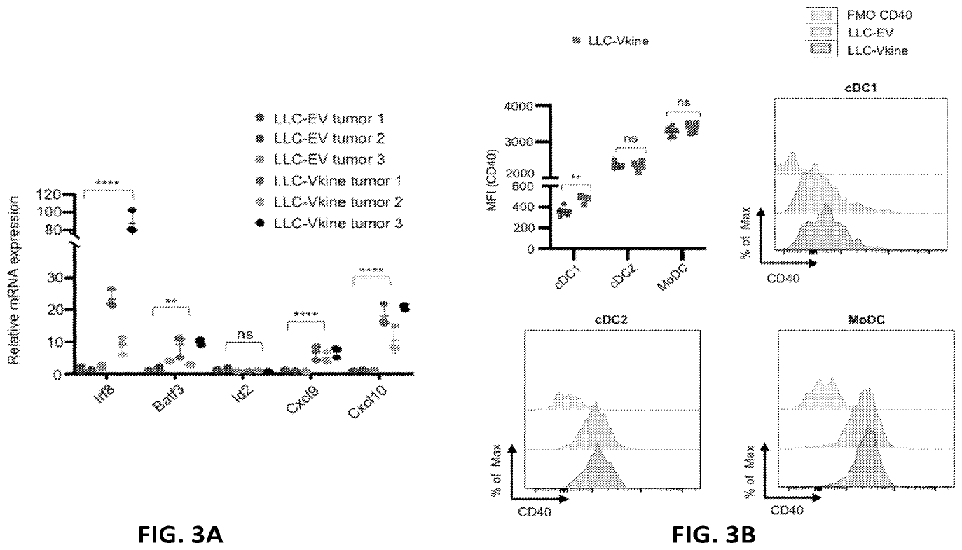

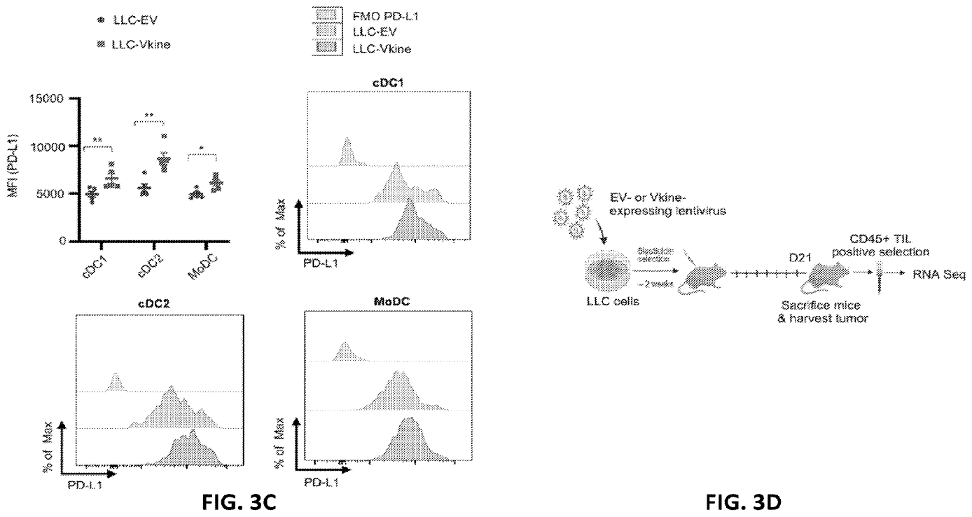

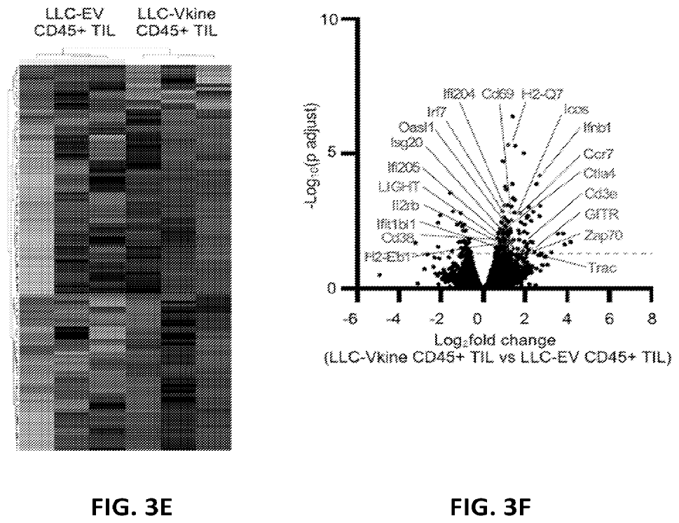

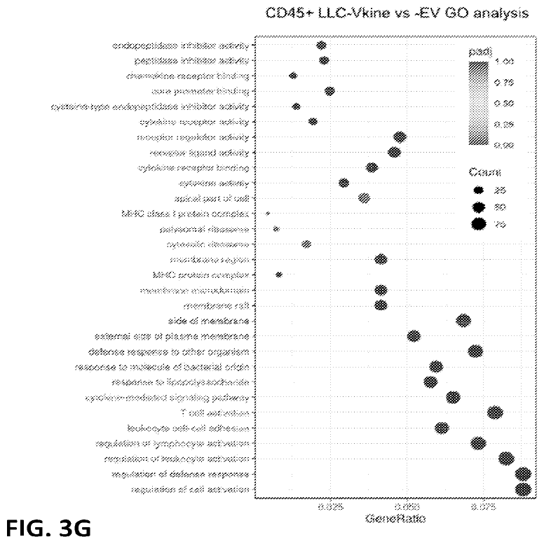

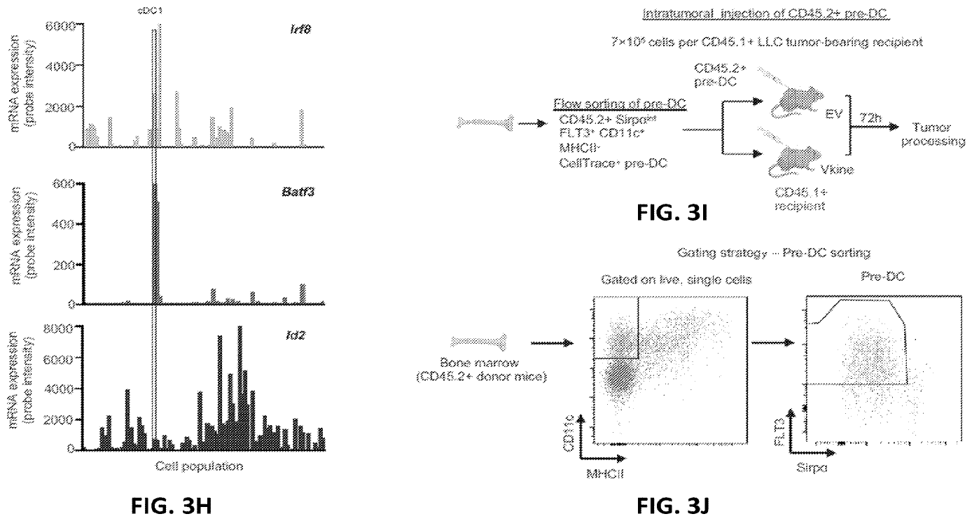

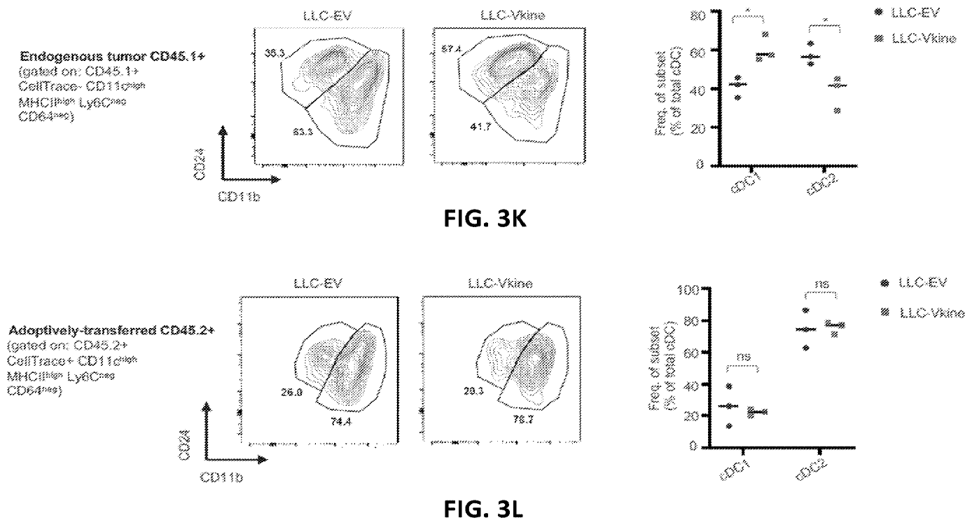

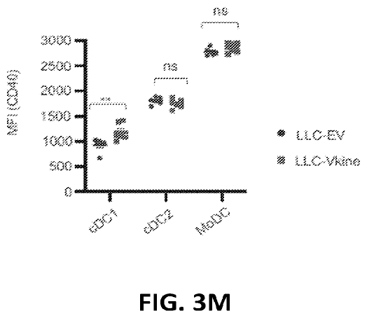

To explain how VCAN proteolytic products promote tumor cDC1 density, it was first determined whether this occurred through uncommitted tumor-seeding pre-DC differentiation. The rationale was based on prior observation that recombinant versikine promoted cDC1 generation from mouse BM treated with FLT3L in vitro. cDC1 “signature” transcripts (Irf8, Batf3, Cxcl9, and Cxcl10) were increased in the bulk transcriptome of versikine-replete (LLC-Vkine) tumors (FIG. 3A). Irf8 is a “terminal selector” for the cDC1 lineage. The Batf3 transcript increase corroborates versikine-induced cDC1 abundance because the Batf3 expression range is very narrow (FIG. 3H). Id2 transcripts did not differ between versikine-replete and control tumors, but Id2 is expressed more broadly and not highly expressed in cDC1s (FIG. 3H).

To test the differentiation hypothesis, CD45.2+ pre-DC precursors were sorted from the BM of Flt3l in-vivo-mobilized donor mice (FIGS. 3I-3J). Donor mice were implanted with Flt3l-secreting B16 tumor cells to provide a continuous source of circulating Flt3l. CD45.2+ pre-DCs were adoptively transferred intratumorally into subcutaneous LLC-EV and LLC-Vkine tumors implanted into CD45.1+ recipients. 72 h after adoptive transfer, tumors were dissociated, and CD45.2+ as well as endogenous CD45.1+DC fractions were enumerated by flow cytometry. The CD45.1+ endogenous cDC composition served as an internal control. As expected, CD45.1+ endogenous cDC1s were increased in LLC-Vkine tumors (FIG. 3K). In contrast, CD45.2+cDC1s and cDC2s did not differ between LLC-Vkine and -EV controls (FIG. 3L). Thus, stromal signals did not affect pre-DC differentiation into cDC1s versus cDC2s within the time frame of the differentiation assay and given the assay's limitations.

### Example 4—Versikine Selectively Activates cDC1s In Vivo

Because pre-DC differentiation could not explain cDC1 accumulation, the hypothesis that versikine regulates the cDC1 activation-survival cycle was tested. The rationale was supported by the association between VCAN proteolysis and T cell infiltration, suggesting a role of versikine in cDC1 activation. CD40 expression was specifically observed because of recent work showing that cDC1s cross-prime CD4+ T cells and are licensed through CD40 back-signaling to augment CD8+ T cell priming and anti-tumor responses as well as the reported anti-apoptotic functions of CD40 in cDC1s. Flow cytometry analysis of cDC subsets from LLC-EV and LLC-Vkine tumors demonstrated that versikine selectively upregulates CD40 expression in tumor cDC1s but not tumor cDC2s or Mo-DCs (FIGS. 3B and 3M). CD40 expression is weakest at baseline in intratumoral cDC1s. In contrast, all 3 intratumoral DC subsets upregulate PD-L1 when exposed to versikine in vivo (FIG. 3C).

CD40 induction in cDC1s would be expected to promote T cell activation in the TME. To test this hypothesis, the transcriptomic profiles of CD45+ tumor-infiltrating leukocytes (TILs) isolated from versikine-replete versus control LLC tumors were compared (FIGS. 3D-3G). A compelling T cell co-stimulation and activation signature was detected (Cd69, Ctla-4, Icos, Zap70, Il2rb, Cd38, Light, and Gitr) (FIGS. 3F and 3G) as well as a significant increase in T cell-specific transcripts (CD3e and T cell receptor [TCR] genes), consistent with the CD8+ T cell expansion seen by mass cytometry (FIG. 2F). Hallmarks of antigen-presenting cell (APC) activation were detected (upregulation of MHCII, Ccr7, Ifnb1, Irf7, and several interferon-responsive genes) (FIG. 3F).

### Example 5—cDC1 Activation by Versikine is Cell Autonomous

To explore steady-state changes in the MutuDC1940 transcriptome in the presence of versikine, stable MutuDC1940-Vkine cell lines were generated through lentiviral transduction (FIG. 4A). MutuDC1940-Vkine cells had a slightly more developed dendritic appearance compared with EV (FIG. 4B).

Versikine elicited a co-stimulatory transcriptional program in MutuDC1940 cells distinct from the transcriptional program elicited by the TLR4 agonist LPS (FIGS. 4C and 4L). The combination of versikine and LPS elicited a transcriptional signature distinct from either stimulus alone (FIG. 4C). Versikine-upregulated genes involved in DC maturation (interferon-stimulated genes such as Ifi209 and Ifi204), chemokines (Ccl7, Ccl2, Cxcl9, and Cxcl10), and co-stimulatory signals (Cd80 and Cd40) (FIG. 4D). Downregulated genes included components of transforming growth factor b (TGF-b) and Wnt pathways, both associated with immunosuppression. Gene set enrichment analysis (GSEA) confirmed upregulation of immune activation gene sets (e.g., interferon [IFN]-a response, IFN-g signaling, nuclear factor kB [NF-kB]-induced tumor necrosis factor [TNF] signaling, and inflammation) and downregulation of immunosuppressive Wntb-catenin and TGF-b signaling (FIG. 4E). Several of the top hits were confirmed by RT-PCR and, at the protein level, by ELISA (FIGS. 4M-4P). Key hits were confirmed after exposure of MutuDC1940 cells to supernatants from HEK293 cells secreting versikine (FIG. 4Q). One of the top hits, Ccl7, has been shown recently to act as a cDC1 chemoattractant. LLC-Vkine tumors expressed high bulk Ccl7 transcripts (FIG. 4F), as did immunomagnetically separated CD11c+ cells from LLC-Vkine tumors (FIG. 4G).

Then, to test versikine's effect on T cell priming by cDC1s, antigen presentation assays were carried out using the OVA (ovalbumin) antigen system in conjunction with TCR-engineered OT-I T cells (FIG. 4H). Vkine- and EV-MutuDC1940 cells were pulsed with SIINFEKL peptides and cocultured with OT-J cells (FIGS. 4I-4K and 4R-4T). Versikine alone more than doubled the percentage of primed OT-I cells secreting IFN-g and interleukin-2 (IL-2) by flow cytometry, confirmed through IFN-g ELISA of culture supernatants. Combining versikine with LPS further augmented T cell priming. Thus, versikine synergized with classical “danger” signals to maximize stimulatory DC antigen presentation and T cell priming.

### Example 6—cDC1 Accumulation Requires Atypical Innate Lymphoid Support

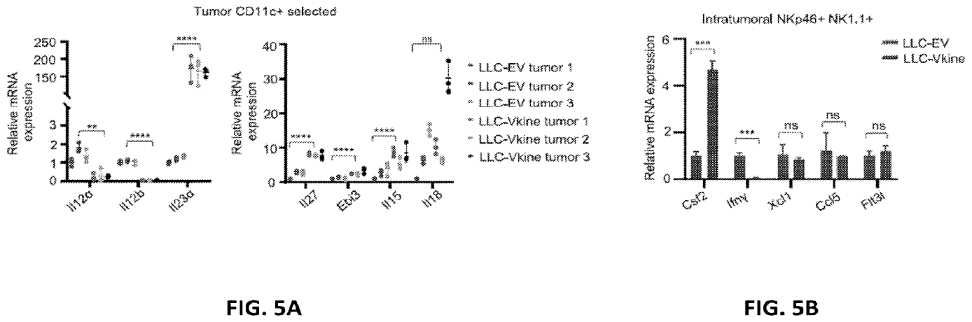

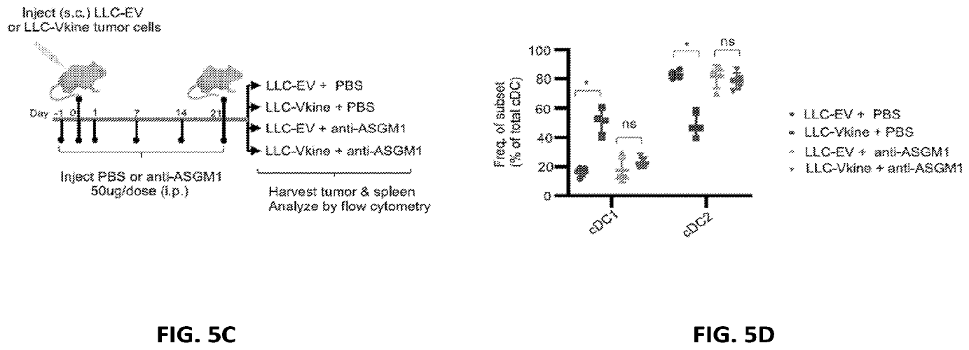

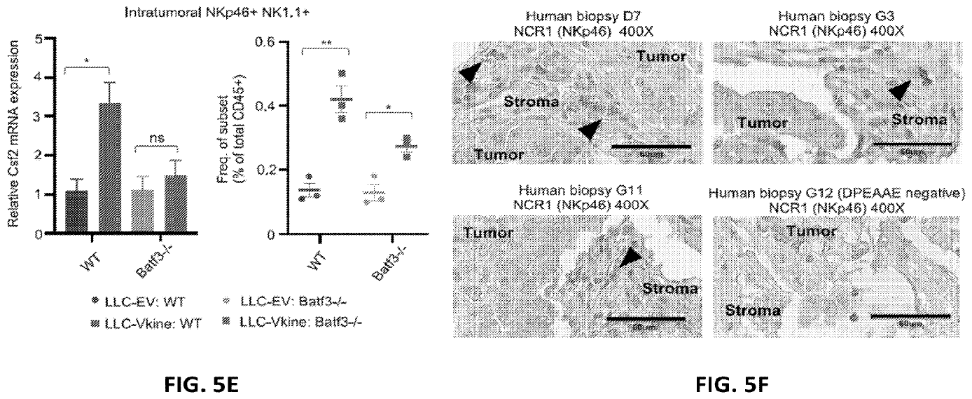

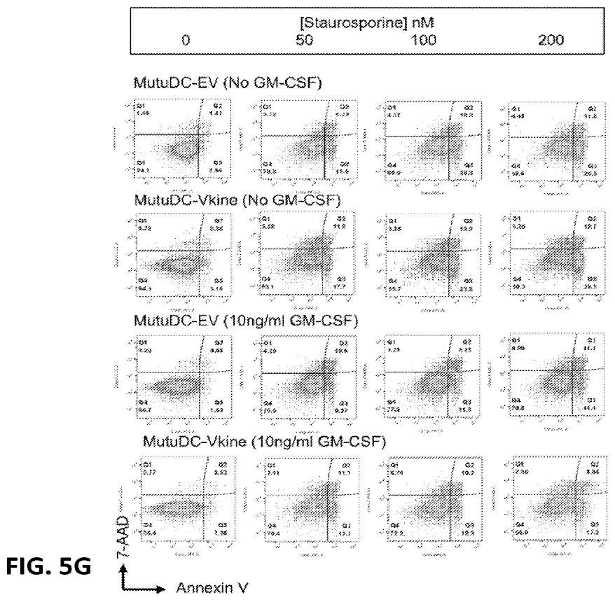

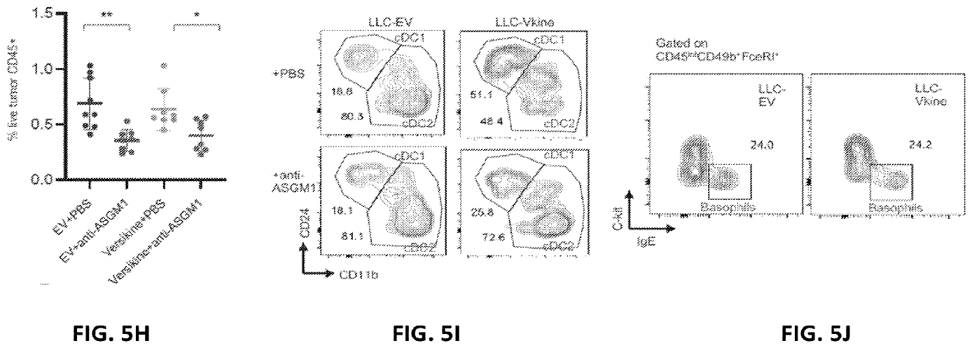

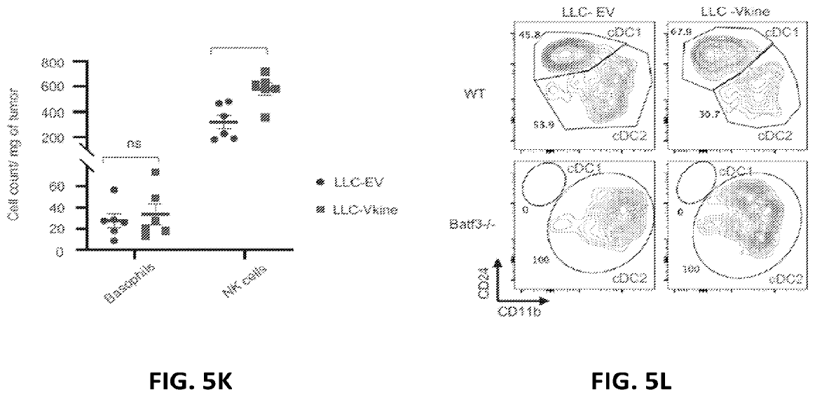

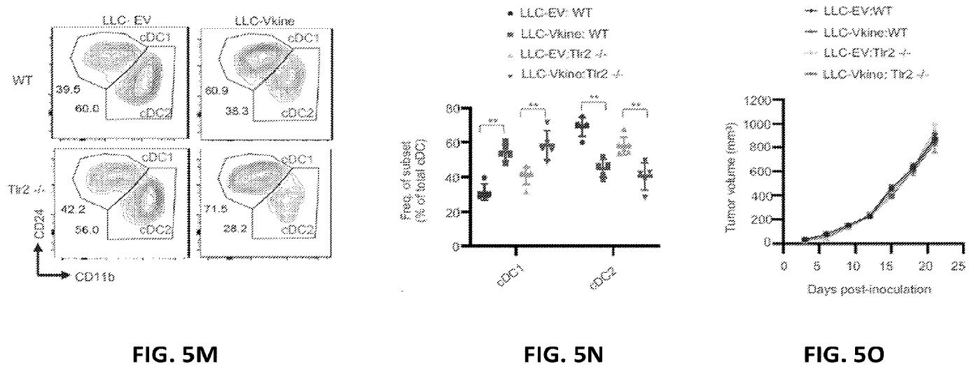

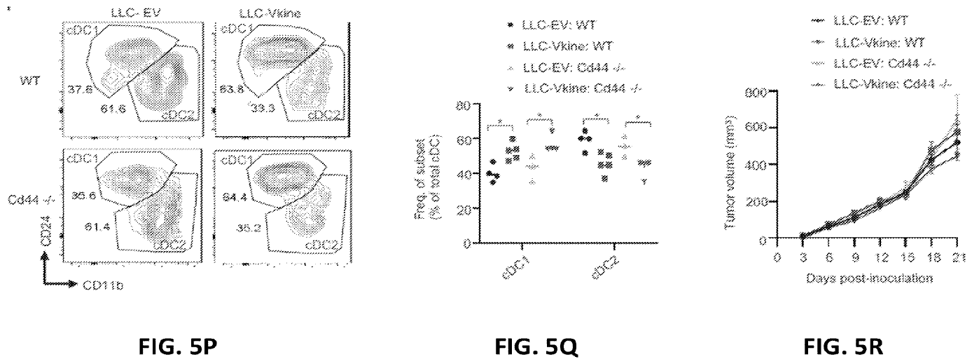

Although cDC1 activation by versikine was cell autonomous, cDC1 accumulation might still require supporting actors, such as natural killer (NK) cells. To determine whether versikine regulates cDC1/NK cell cross-talk in vivo, CD11c+ DCs from primary versikine-replete versus EV tumors were characterized. Freshly explanted CD11c+ cells from LLC-Vkine tumors expressed higher levels of the NK regulators IL-23 (a subunit), IL-27 (p28 and EBI3 subunits), and IL-15 (FIG. 5A). Therefore, versikine's cDC1 activation program incorporated an NK cell-activating module. Versikine-exposed DCs demonstrated low IL-12a(p35) and IL-12b(p40) subunit expression but very high IL-23a expression. It was speculated that IL-23a could combine with IL-12b(p40) secreted by other activated myeloid cells (e.g., macrophages) to form bioactive IL-23 heterodimers, similar to extracellular IL-12p70 generation.

It was hypothesized that versikine may trigger a distinct stroma-specific pattern of cDC1-NK cell communication. Previous studies have implicated the NK cell-derived differentiation/survival mediator FLT3L as well as the chemo-attractants XCL1 and CCL5 in cDC1 support. NK cell-derived IFN-g has been shown recently to induce the cDC1 “terminal selector” IRF8. NKp46+NK1.1+ cells from LLC-Vkine tumors were potent expressors of Csf2 (granulocyte-macrophage colony stimulating factor [GM-CSF]) and relatively weak expressors of IFN-g compared with the LLC-EV-derived counterparts (FIG. 5B), whereas expression of Xc1l, Flt3l, and Ccl5 remained unchanged (FIG. 5B). Versikine causes induction/expansion of an atypical NK cell subset expressing low IFN-g despite high cytotoxicity receptor (NKp46) expression and robust Csf2 (GM-CSF), an essential survival factor for cDC1s. These results suggest that versikine engages innate lymphoid cells through a previously unreported mechanism in tumors. Versikine-induced NK cells are reminiscent of a recently reported spleen-resident ILC1-like subset that nurses cDC1s and promotes CD8+ T cell priming in viral infection.

To determine whether versikine—requires innate lymphoid cells to support cDC1 abundance in the TME, an antiasialo-GM1 antibody (anti-ASGM1) was used for in vivo NK cell depletion (FIG. 5C). Anti-ASGM1 antibody treatment (FIG. 5H) completely abrogated versikine-mediated enhancement of cDC1s in the TME (FIGS. 5D and 5I).

### Example 7—Versikine Regulates cDC1-NK Cell Cross-Talk Through cDC1s

The results so far suggested that versikine regulates cDC1s and NK cells in a virtuous circle but left open the question of whether versikine's primary target is cDC1s or NK cells. To answer this question, the experiment was repeated in Batf3^{-/-} mice, which lack intratumoral cDC1s (FIG. 5L). In Batf3^{-/-} recipients, versikine was unable to induce Csf2 (GMCSF) in intratumoral NKp46+NK1.1+ cells despite a modest increase in NK cell frequency (FIG. 5E). This result suggested that the primary target of versikine in the cDC1-NK cell cycle was cDC1s. This model would pre-suppose proximity of cDC1s and NKp46+ cells inhuman tumor stroma. NCR1 (NKp46+) cells in human tumors were almost exclusively stromal in their location (FIG. 5F).

The evidence so far supports a model where versikine acts directly on stromal cDC1s to activate a pro-immunogenic program. Like other known cDC1 activators (e.g., TLR ligands), versikine-mediated activation simultaneously triggers a homeostatic propensity for apoptosis. This pro-apoptotic propensity is mitigated/rescued by NK cell-derived GM-CSF. The end result is accumulation of activated cDC1s (which can be called stroma-licensed cDC1s) in stroma undergoing VCAN proteolysis. Consistent with this model, versikine's MutuDC1940 signature incorporates a pro-apoptotic module (FIG. 4L). Experimentally, it was confirmed that versikine-expressing MutuDC1940 cells are sensitive to apoptotic signals and that this sensitivity is mitigated/rescued by exogenous murine GM-CSF (FIG. 5G). In the TME, NK cells are dominant GM-CSF producers compared with sparser GM-CSF expressors, such as basophils (FIGS. 5J and 5K).

### Example 8—TLR2 and CD44 are Dispensable for cDC1 Accumulation in Response to Versikine

The data are consistent with versikine signaling through a DC receptor whose identity is unknown. Although cDC1s do not robustly express TLR2, non-proteolyzed VCAN is thought to act through TLR2 expressed broadly by DC subsets. However, Tlr2 loss had no effect on versikine-induced cDC1 accumulation (FIGS. 5M-5O).

It was also considered that versikine may activate hyaluronan-dependent signaling pathways in vivo. VCAN's N-terminal link domains bind hyaluronan (FIG. 1A). Recombinant versikine bioactivity was previously demonstrated in the absence of bound hyaluronan in vitro. Loss of Cd44, encoding the major hyaluronan receptor, did not affect versikine-induced cDC1 accumulation in vivo (FIGS. 5P-5R).

### Example 9—Stroma-Licensed cDC1s are “Poised” and Hypersensitive to Nucleic Acid sensing in vivo

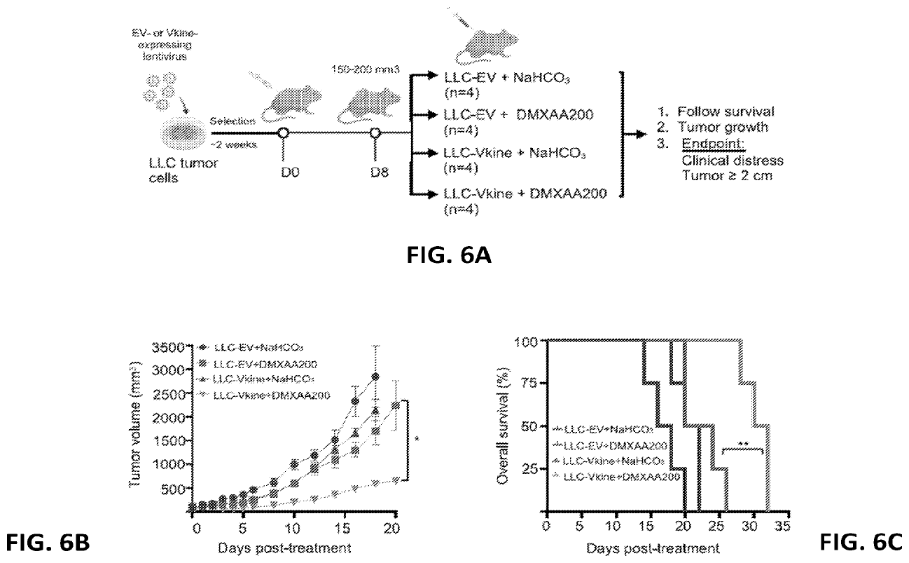

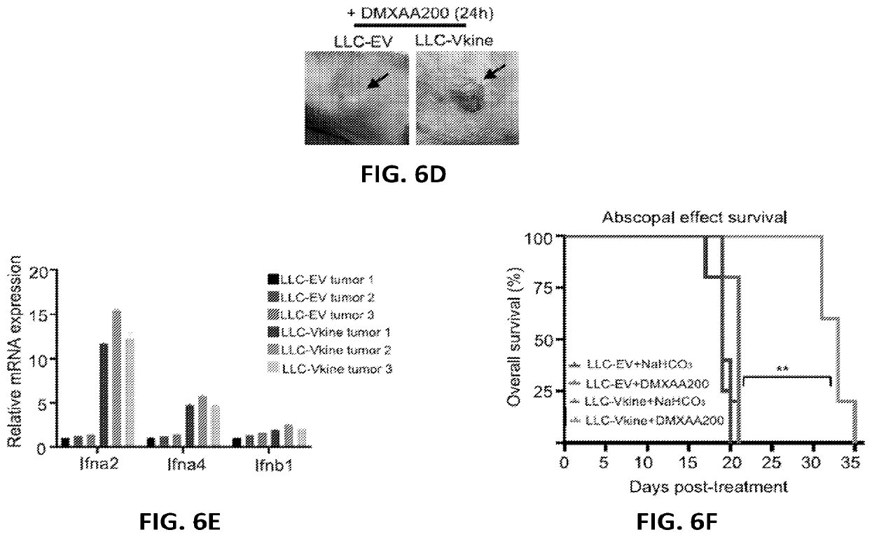

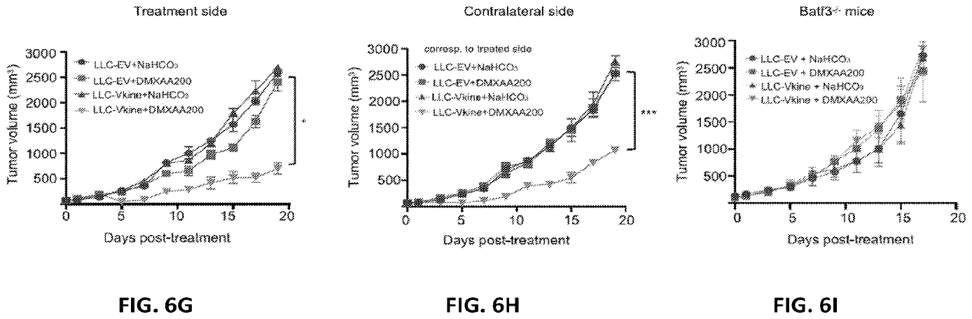

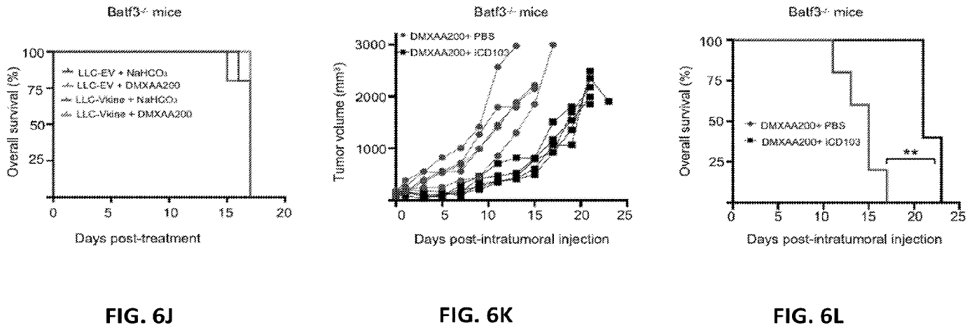

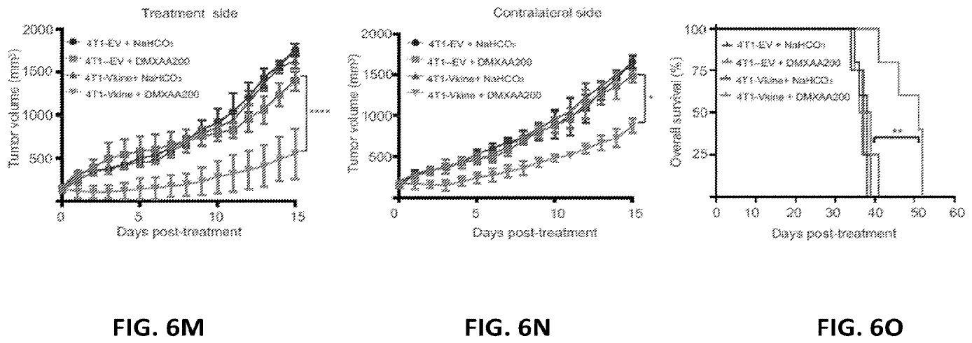

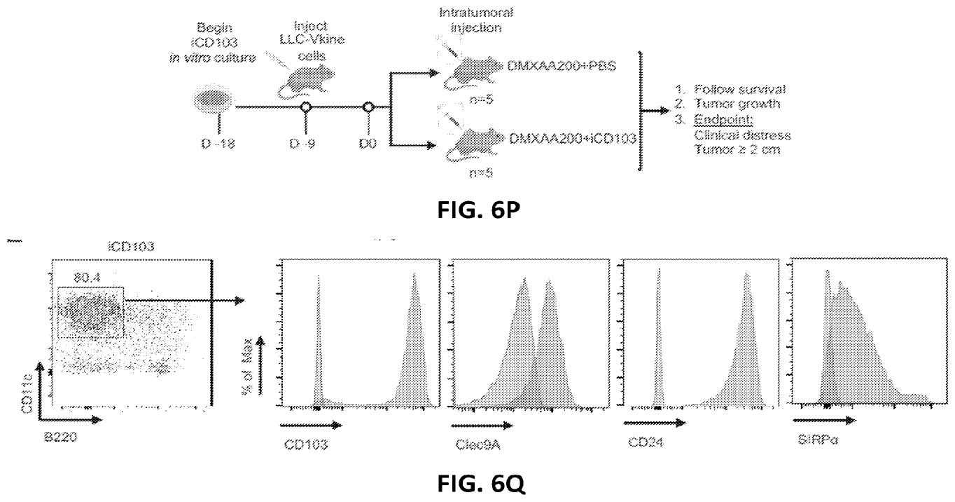

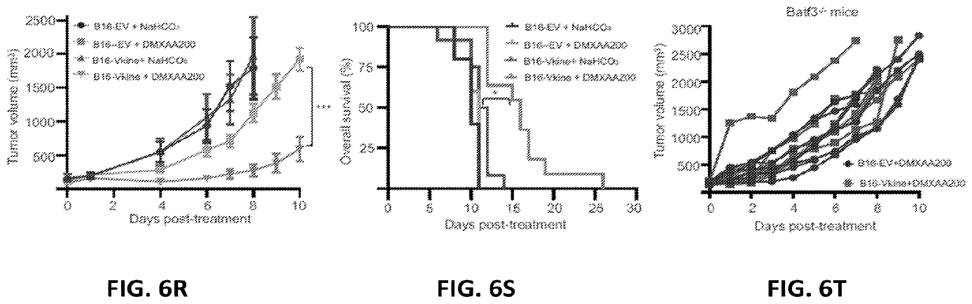

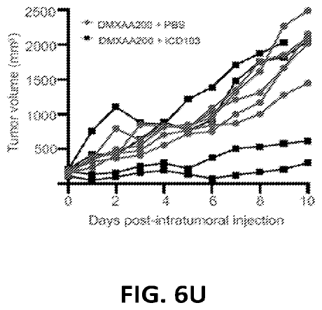

Previous studies have highlighted the paradox of immunogenic DC accumulation along the tumor rim, but no compelling mechanism has emerged. It was hypothesized that peritumoral stroma-licensed DCs are “poised” to respond to physiological maturation signals arising from necrotic tumor cells. Thus, versikine-induced CD40 expression and “stroma licensing” could serve to amplify these innate cancer-sensing signals. The cGAS/STING pathway, a sensor of exogenous double-stranded DNA, has emerged as a central mediator of innate sensing of tumors. It was hypothesized that stroma-licensed DCs may respond to very low doses of a STING agonist (FIG. 6A). Dose-response relationships were previously established for the intratumorally administered murine STING agonist DMXAA: the maximum tolerated dose (MTD) was 500 mg; unacceptable toxicity was observed at higher doses. LLC-EV and LLC-Vkine tumors were challenged with subtherapeutic doses of the STING agonist DMXAA (150-200 mg) or vehicle (NaHCO₃). Tumor response curves are shown in FIG. 6B and survival plots (Kaplan-Meier) in FIG. 6C. EV tumors did not appreciably respond to vehicle or subtherapeutic doses of DMXAA (DMXAA200). In contrast, versikine lowered the response threshold to DMXAA so that versikine-replete tumors demonstrated a consistent response to single subtherapeutic DMXAA doses. Versikine-replete tumors routinely developed necrotic eschars by 24 h after subtherapeutic DMXAA injection (FIG. 6D). In contrast, none of the control mice developed eschars within this time frame. To determine whether versikine reduced the therapeutic threshold through a classic type I IFN response to DMXAA, tumors for RNA extraction were harvested 2 h after DMXAA administration. Versikine-replete tumors demonstrated a several-fold increase in IFN-α transcripts (particularly IFN-α2 and IFN-α4) and, to a lesser degree, IFN-01 transcripts (FIG. 6E). These results demonstrate that versikine lowered the threshold for a classic type I IFN-mediated STING agonist response.

To determine whether low doses of the STING agonist could generate an abscopal effect, mice bearing tumors implanted in both flanks were examined. The treated side was inoculated with EV- or versikine-expressing LLC cells; the contralateral, nontreated side was inoculated with unmanipulated (untransduced) LLC cells. Ectopic versikine is bound in the pericellular halo (glycocalyx) (FIG. 2D) and probably does not circulate to an appreciable degree. A consistent abscopal effect was observed when versikine-replete tumors were injected with 200 mg DMXAA (FIGS. 6F-6H). EV tumors treated with the same subtherapeutic dose failed to elicit any response on the treatment or contralateral side. STING agonist hypersensitivity produced consistent primary tumor and abscopal effects across genetic backgrounds; e.g., in the orthotopic 4T1 mammary carcinoma model (FIGS. 6M-6O).

The hypothesis that stromal matrikines render cDC1 hypersensitive to nucleic acid sensing in vivo predicts that DMXAA200 would be ineffective in the absence of cDC1s. Thus, the experiment delineated in FIG. 6A was repeated in Batf3^{-/-} recipients. DMXAA200 was globally ineffective in the Batf3-null background, and all survival benefit was lost (FIGS. 6I and 6J). To confirm that the responsible actors were cDC1s (rather than another Batf3-expressing lineage), it was attempted to rescue the null phenotype with intratumoral adoptive transfer of iCD103, BM-derived primary cDC1-like cells generated in culture (FIG. 6P). MutuDC1940 cells cannot be used for adoptive transfer experiments because of their immunogenicity. A consistent cDC1-like phenotype was confirmed in iCD103 cells (FIG. 6Q). Adoptive transfer of iCD103 cells restored subtherapeutic STING agonist efficacy (FIG. 6K) and survival benefits (FIG. 6L). To confirm the findings in a different C57BL6/J model, the B16 melanoma model was chosen. B16 tumors responded to subtherapeutic doses of DMXAA in the presence of versikine but not EV (FIGS. 6R and 6S). Efficacy was lost in the Batf3-null background (FIG. 6T), but response to subtherapeutic doses of the STING agonists was restored, at least in a subset of mice, upon iCD103 adoptive transfer (iCD103 “take” in B16, an “immune-cold” tumor, was less efficient than in LLC, an “immune-hot” tumor, FIG. 6U). Therefore, stroma-licensed cDC1s are “poised” and hypersensitive to nucleic acid sensing in vivo.

### Example 10—Versikine Promotes Antigen-Specific CD8+ T Cell Responses In Vivo

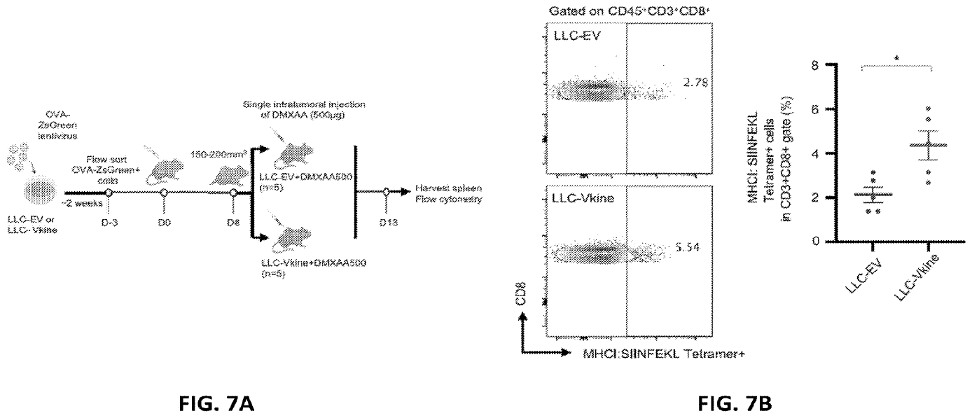

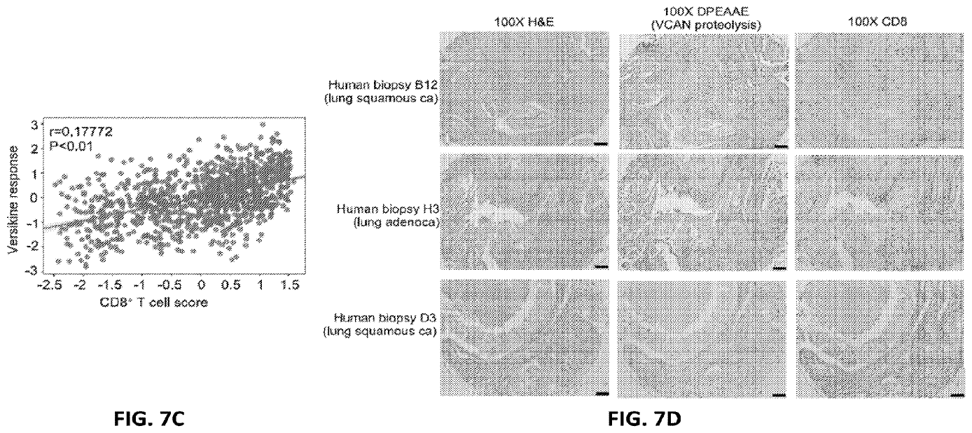

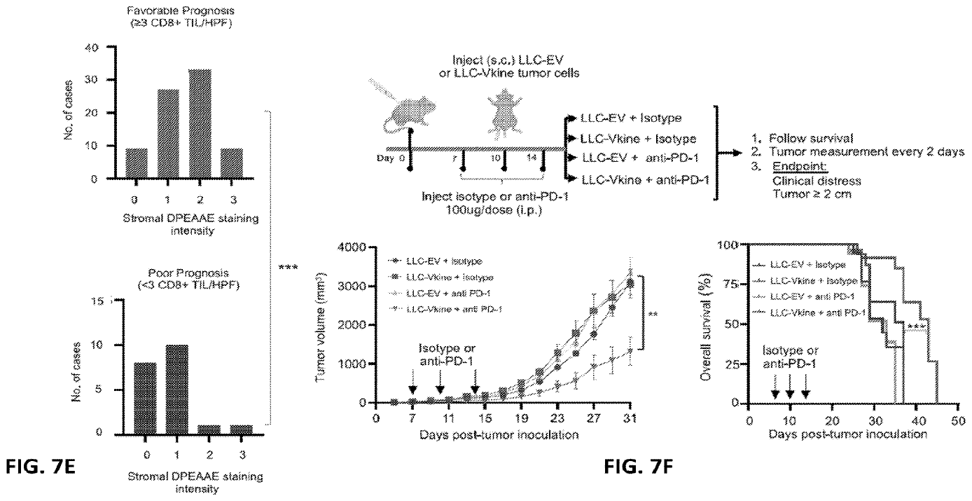

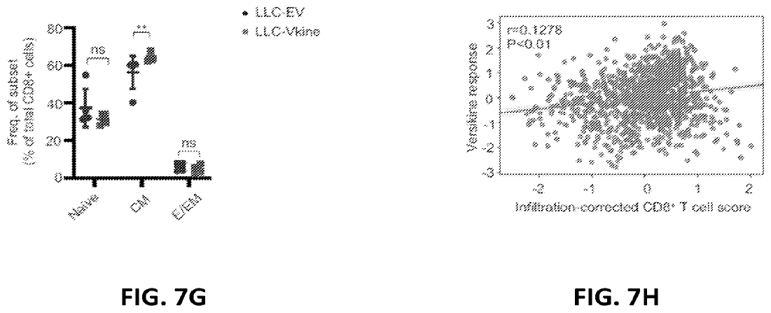

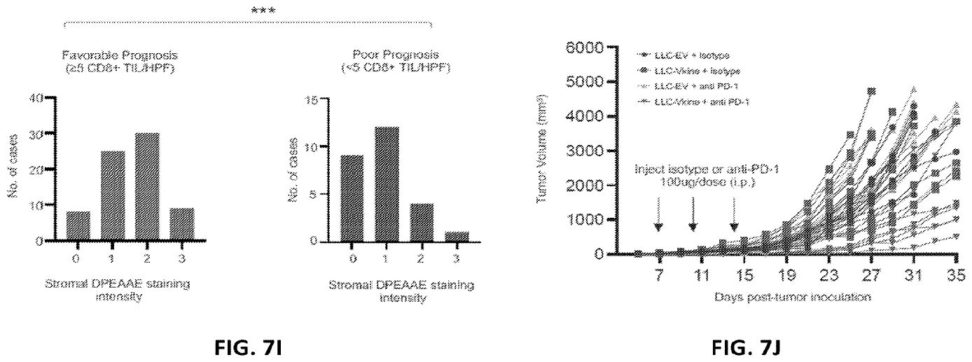

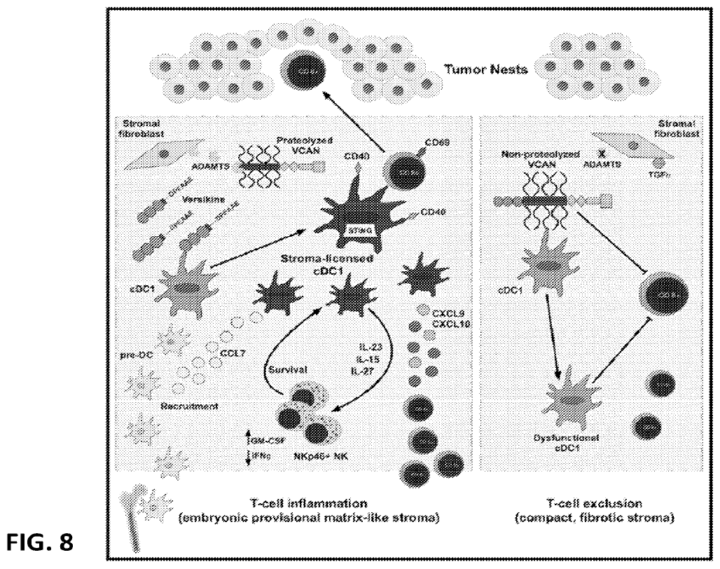

To determine whether hypersensitivity of stromalicensed cDC1s to DNA sensing translates into enhanced antigen-specific effector responses in vivo, the OVA system was employed as an in vivo model antigen (FIG. 7A). EV- and versikine-expressing LLC cells were additionally engineered to express full-length OVA (LLC-OVA). EV- or versikine-replete LLC-OVA tumors were challenged with therapeutic DMXAA doses (500 mg). Five days after challenge, spleens were harvested and analyzed by flow cytometry for antigen-specific effector responses using an antigen-specific tetramer assay. Versikine more than doubled the magnitude of the antigen-specific response in the CD8+ T cell compartment, as determined by MHCI:SIINFEKL-tetramer staining (FIG. 7B). Spleens contained a larger proportion of CD8+CD62L+CD44+ cells with a central memory phenotype (FIG. 7G). The results demonstrate that stromal matrikines enhance antigen-specific CD8+ T cell effector responses in vivo.

### Example 11—the Stroma-Licensed cDC1 Signature Correlates with CD8+ T Cell Scores in Human Lung Cancer

To correlate stromal cDC1 licensing with CD8+ T cell density in human cancers, a unique cDC1 response signature was generated from 200 genes whose expression was significantly altered in versikine-activated MutuDC1940 cells (FIGS. 4C and 4D). Then, this stroma-cDC1 signature was correlated with CD8+ T cell scores estimated in TCGA expression data for 1,017 lung cancers (STAR Methods). The results are shown in FIGS. 7C and 7H. A significant but weak correlation was observed between stroma-licensed cDC1 transcriptional profiles and CD8+ T cell scores. There were obvious limitations in this analysis (application of an in vitro murine cultured cell-generated signature to primary bulk human tumor data), but the results did support a connection between stromal cDC1 licensing and CD8+ T cell density in human cancer.

### Example 12—Stromal VCAN Proteolysis Correlates with CD8+ Infiltration in Human Lung Cancer

CD8+ T cell infiltration has prognostic significance in human lung cancer as well as predictive significance for efficacy of checkpoint inhibitor-based immunotherapy. A cutoff of 3-5 CD8+ T cells/HPF has been used in some studies to designate CD8+ TIL-rich versus-poor tumors bearing a favorable and unfavorable prognosis, respectively. To determine whether stromal VCAN proteolysis was associated with prognostic immune infiltration groups in humans, 98 non-small cell lung cancer (NSCLC) biopsies in our tissue microarray (TMA) were subdivided into pauci-immune (0-2 CD8+ TILs/HPF in stromal and epithelial compartments, n=26) and immune-rich (R3 CD8+ TILs/HPF in stromal or epithelial compartments, n=72). The distribution of stromal DPEAAE staining intensity (0, 1, 2, or 3) was compared between the groups. The results are shown in FIGS. 7D, 7E, and 7I. A statistically significant association was found between stromal YCAN proteolysis intensity and CD8+ T cell infiltration in human NSCLC.

### Example 13—the VCAN Matrikine Versikine Overcomes Resistance to Anti-PD1 Checkpoint Inhibition Immunotherapy In Vivo

cDC1s are critical for responses to checkpoint inhibitors. Thus, the effect of versikine on anti-PD1 responses in the refractory LLC model was tested (FIGS. 7F and 7J). Versikine sensitized at least a subset of LLC tumors to a short course of anti-PD1 checkpoint inhibition-based immunotherapy, resulting in enhanced animal survival.

1.-73. (canceled)

74. A fusion protein comprising a versikine protein having at least 95% identity to SEQ ID NO: 1 and a protein selected from the group consisting of: GM-CSF, CCL5, XCL1, FLT3L, CCL7, CXCL9, CXCL10, IL-12, IL-27, IL-15, IL18, IL-23, IL-2, and IFN-gamma.

75. The fusion protein of claim 74, wherein the protein selected from the group consisting of GM-CSF, CCL5, XCL1, FLT3L, CCL7, CXCL9, CXCL10, IL-12, IL-27, IL-15, IL18, IL-23, IL-2, and IFN-gamma comprises a sequence selected from the group consisting of SEQ ID NO: 8-21.

76. The fusion protein of claim 74, further comprising a secretory peptide.

77. The fusion protein of claim 76, wherein the secretory peptide comprises a sequence of SEQ ID NO: 2-7.

78. The fusion protein of claim 76, wherein the secretory peptide is selected from the group consisting of: human OSM, human immunoglobulin, human chymotrypsinogen, human trypsinogen 2, human IL-2, or human insulin.

79. The fusion protein of claim 74, wherein the versikine protein is generated by proteolysis of VCAN at the Glu⁴⁴¹-Ala⁴⁴² bond.

80. A pharmaceutical composition comprising the fusion protein of claim 74 and a STING agonist.

81. The pharmaceutical composition of claim 80, wherein the STING agonist is DMXAA or RVU-27065.

82. A pharmaceutical composition comprising the fusion protein of claim 74 and an anti-PD1 and/or anti-PD-L1 inhibitor.

83. The pharmaceutical composition of claim 82, wherein the anti-PD1 and/or anti-PD-L1 inhibitor comprises pembrolizumab, nivolumab, cemiplimab, JTX-4014, spartalizumab, camrelizumab, sintilimab, tislelizumab, toripalimab, dostarlimab, atezolizumab, avelumab, durvalumab, KN035, CK-301, AUNP12, CA-170, or BMS-986189.

84. A pharmaceutical composition comprising the fusion protein of claim 74 and anti-CD40 immunotherapy.

85. The pharmaceutical composition of claim 84, wherein the anti-CD40 immunotherapy is selected from the group consisting of selicrelumab, APX005M, JNJ-64457107, SEA-CD40, ChiLob7/4, CDX-1140H, Dacetuzumab, and ABBV-428.

86. A conjugated polypeptide comprising the fusion protein of claim 74 conjugated to a small molecule STING agonist.

87. The conjugated polypeptide of claim 86, wherein the small molecule STING agonist comprises DMXAA or RVU-27065.

88. A vector comprising a nucleic acid sequence encoding the fusion protein of claim 74.

89. The vector of claim 88, wherein the vector is a viral vector.

90. The vector of claim 89, wherein the viral vector is a lentiviral vector.

91. A method of introducing the versikine protein to antigen-presenting cells, the method comprising introducing to an antigen-presenting cell the vector of claim 90.

92. A method of treating cancer, the method comprising administering a versikine protein having at least 95% sequence identity to SEQ ID NO: 1 or a versikine nucleic acid having at least 95% sequence identity to SEQ ID NO: 23.

93. A method of predicting a response of a subject to an immunotherapy, the method comprising: analyzing a biological sample obtained from the subject; and detecting a proteolytic fragment of an extracellular matrix (ECM) proteoglycan in the biological sample, thereby predicting the subject's response to the immunotherapy.


SEQUENCE LISTING (34 sequences)

SEQ ID NO: 1 - protein, 421 aa, synthetic construct
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKRRMSDLSVIGHPIDSESKEDEPCSEETDPVHDLMAEILPEFPDIIEIDLYHSEENEEEEEECANATDVTTTPSVQYINGKHLVTTVPKDPEAAE

SEQ ID NO: 2 - protein, 25 aa, Homo sapiens
MGVLLTQRTLLSLVLALLFPSMASM

SEQ ID NO: 3 - protein, 22 aa, Homo sapiens
MDMRVPAQLLGLLLLWLRGARC

SEQ ID NO: 4 - protein, 18 aa, Homo sapiens
MAFLWLLSCWALLGTTFG

SEQ ID NO: 5 - protein, 15 aa, Homo sapiens
MNLLLILTFVAAAVA

SEQ ID NO: 6 - protein, 20 aa, Homo sapiens
MYRMQLLSCIALSLALVTNS

SEQ ID NO: 7 - protein, 24 aa, Homo sapiens
MALWMRLLPLLALLALWGPDPAAA

SEQ ID NO: 8 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 9 - protein, 91 aa, Homo sapiens [sequence omitted]

SEQ ID NO: 10 - protein, 114 aa, Homo sapiens
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 11 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 12 - protein, 99 aa, Homo sapiens
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 13 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 14 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 15 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 16 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 17 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 18 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 20 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 21 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 22 - protein, 20 aa, synthetic construct
MFINIKSILWMCSTLIVTHA

SEQ ID NO: 23 - DNA, 1323 bp, synthetic construct
  source 1..1323: mol_type = other DNA
atgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacgtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaa

SEQ ID NO: 24 - protein, 4 aa, synthetic construct
GGGS

SEQ ID NO: 25 - protein, 12 aa, synthetic construct
GGGSGGGSGGGS

SEQ ID NO: 26 - protein, 8 aa, Synthetic construct
GGGGGGGG

SEQ ID NO: 27 - protein, 15 aa, Synthetic construct
EAAAKEAAAKEAAAK

SEQ ID NO: 28 - protein, 5 aa, Synthetic construct
PAPAP

SEQ ID NO: 29 - protein, 12 aa, Synthetic construct
AEAAAKEAAAKA

SEQ ID NO: 30 - protein, 18 aa, Synthetic construct
KESGSVSSEQLAQFRSLD

SEQ ID NO: 31 - protein, 14 aa, synthetic construct
EGKSSGSGSESKST

SEQ ID NO: 32 - protein, 12 aa, Synthetic construct
GSAGSAAGSGEF

SEQ ID NO: 33 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 34 - protein, 188 aa, Synthetic construct
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE